SEQUENCE LISTING (9 sequences)

SEQ ID NO: 1 - DNA, 1509 bp, Artificial sequence
  expression module for human MG53 in plasmid of SEQ ID NO 4
cctaggatgaaaaaaaagattatctcagctattttaatgtctacagtgatactttcggctgcgcccggcctcctgcaccaggagctgtcctgcccgctgtgcctgcagctgttcgacgcgcccgtgacagccgagtgcggccacagtttctgccgcgcctgcctaggccgcgtggccggggagccggcggcggatggcaccgttctctgcccctgctgccaggcccccacgcggccgcaggcactcagcaccaacctgcagctggcgcgcctggtggaggggctggcccaggtgccgcagggccactgcgaggagcacctggacccgctgagcatctactgcgagcaggaccgcgcgctggtgtgcggagtgtgcgcctcactcggctcgcaccgcggtcatcgcctcctgcctgccgccgaggcccacgcacgcctcaagacacagctgccacagcagaaactgcagctgcaggaggcatgcatgcgcaaggagaagagtgtggctgtgctggagcatcagctggtggaggtggaggagacagtgcgtcagttccggggggccgtgggggagcagctgggcaagatgcgggtgttcctggctgcactggagggctccttggaccgcgaggcagagcgtgtacggggtgaggcaggggtcgccttgcgccgggagctggggagcctgaactcttacctggagcagctgcggcagatggagaaggtcctggaggaggtggcggacaagccgcagactgagttcctcatgaaatactgcctggtgaccagcaggctgcagaagatcctggcagagtctcccccacccgcccgtctggacatccagctgccaattatctcagatgacttcaaattccaggtgtggaggaagatgttccgggctctgatgccagcgctggaggagctgacctttgacccgagctctgcgcacccgagcctggtggtgtcttcctctggccgccgcgtggagtgctcggagcagaaggcgccgccggccggggaggacccgcgccagttcgacaaggcggtggcggtggtggcgcaccagcagctctccgagggcgagcactactgggaggtggatgttggcgacaagccgcgctgggcgctgggcgtgatcgcggccgaggccccccgccgcgggcgcctgcacgcggtgccctcgcagggcctgtggctgctggggctgcgcgagggcaagatcctggaggcacacgtggaggccaaggagccgcgcgctctgcgcagccccgagaggcggcccacgcgcattggcctttacctgagcttcggcgacggcgtcctctccttctacgatgccagcgacgccgacgcgctcgtgccgctttttgccttccacgagcgcctgcccaggcccgtgtaccccttcttcgacgtgtgctggcacgacaagggcaagaatgcccagccgctgctgctcgtgggtcccgaaggcgccgaggcccaccaccaccatcaccactgaggtacc

SEQ ID NO: 2 - DNA, 18 bp, Artificial Sequence
  upstream primer
caccgttctctgcccctg

SEQ ID NO: 3 - DNA, 18 bp, Artificial sequence
  downstream primer
ctgtgtcttgaggcgtgc

SEQ ID NO: 4 - DNA, 4649 bp, Artificial sequence
  SP-MG53-6xHis in the pNZ8148 plasmid
agatctagtcttataactatactgacaatagaaacattaacaaatctaaaacagtcttaattctatcttgagaaagtattggtaataatattattgtcgataacgcgagcataataaacggctctgattaaattctgaagtttgttagatacaatgatttcgttcgaaggaactacaaaataaattataaggaggcactcacctaggatgaaaaaaaagattatctcagctattttaatgtctacagtgatactttcggctgcgcccggcctcctgcaccaggagctgtcctgcccgctgtgcctgcagctgttcgacgcgcccgtgacagccgagtgcggccacagtttctgccgcgcctgcctaggccgcgtggccggggagccggcggcggatggcaccgttctctgcccctgctgccaggcccccacgcggccgcaggcactcagcaccaacctgcagctggcgcgcctggtggaggggctggcccaggtgccgcagggccactgcgaggagcacctggacccgctgagcatctactgcgagcaggaccgcgcgctggtgtgcggagtgtgcgcctcactcggctcgcaccgcggtcatcgcctcctgcctgccgccgaggcccacgcacgcctcaagacacagctgccacagcagaaactgcagctgcaggaggcatgcatgcgcaaggagaagagtgtggctgtgctggagcatcagctggtggaggtggaggagacagtgcgtcagttccggggggccgtgggggagcagctgggcaagatgcgggtgttcctggctgcactggagggctccttggaccgcgaggcagagcgtgtacggggtgaggcaggggtcgccttgcgccgggagctggggagcctgaactcttacctggagcagctgcggcagatggagaaggtcctggaggaggtggcggacaagccgcagactgagttcctcatgaaatactgcctggtgaccagcaggctgcagaagatcctggcagagtctcccccacccgcccgtctggacatccagctgccaattatctcagatgacttcaaattccaggtgtggaggaagatgttccgggctctgatgccagcgctggaggagctgacctttgacccgagctctgcgcacccgagcctggtggtgtcttcctctggccgccgcgtggagtgctcggagcagaaggcgccgccggccggggaggacccgcgccagttcgacaaggcggtggcggtggtggcgcaccagcagctctccgagggcgagcactactgggaggtggatgttggcgacaagccgcgctgggcgctgggcgtgatcgcggccgaggccccccgccgcgggcgcctgcacgcggtgccctcgcagggcctgtggctgctggggctgcgcgagggcaagatcctggaggcacacgtggaggccaaggagccgcgcgctctgcgcagccccgagaggcggcccacgcgcattggcctttacctgagcttcggcgacggcgtcctctccttctacgatgccagcgacgccgacgcgctcgtgccgctttttgccttccacgagcgcctgcccaggcccgtgtaccccttcttcgacgtgtgctggcacgacaagggcaagaatgcccagccgctgctgctcgtgggtcccgaaggcgccgaggcccaccaccaccatcaccactgaggtaccactagttctagagagctcaagctttctttgaaccaaaattagaaaaccaaggcttgaaacgttcaattgaaatggcaattaaacaaattacagcacgtgttgctttgattgatagccaaaaagcagcagttgataaagcaattactgatattgctgaaaaattgtaatttataaataaaaatcaccttttagaggtggtttttttatttataaattattcgtttgatttcgctttcgatagaacaatcaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgtccttctatttcggttggaggaggctcaagggagtttgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttgaggggtttttcaatcgccaacgaatcgccaacgttttcgccaacgttttttataaatctatatttaagtagctttatttttgtttttatgattacaaagtgatacactaattttataaaattatttgattggagttttttaaatggtgatttcagaatcgaaaaaaagagttatgatttctctgacaaaagagcaagataaaaaattaacagatatggcgaaacaaaaagatttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagcccccctgacgaaagtcgacggcaatagttacccttattatcaagataagaaagaaaaggatttttcgctacgctcaaatcctttaaaaaaacacaaaagaccacattttttaatgtggtcttttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccttggacttcatttactgggtttaacttaaatatcaataataatagtaattaccttctacccattattacagcaggaaaattcattaataaaggtaattcaatatatttaccgctatctttacaggtacatcattctgtttgtgatggttatcatgctggattgtttatgaactctattcaggaattgtcagataggcctaatgactggcttttataatatgagataatgccgactgtactttttacagtcggttttctaatgtcactaacctgccccgttagttgaagaaggtttttatattacagctcc

SEQ ID NO: 5 - DNA, 1569 bp, Artificial Sequence
  expression module of MG53 as part of plasmid in SEQ ID NO 4
ggcgagctcaaatgcaacgtaaaaagaaaggtttaagtatcttattagctggtacagttgctttaggtgctttagctgttttaccagttggcgaaatccaagcaaaggctgacacaaacagtgatatgagtgctgctccaggcttattacaccaggaattaagttgcccgttatgcttacagttattcgacgctccagttacagctgaatgcggccacagtttctgccgtgcttgcttaggccgtgttgctggtgaaccggctgctgatggcaccgttttatgcccatgctgccaggctccaacccgtccgcaggcattaagtaccaacttacagttagctcgtttagttgaaggtttagctcaggttccgcagggccactgcgaagaacacttagacccgttaagtatctactgcgaacaggaccgtgctttagtttgcggtgtttgcgcttcattaggcagtcaccgtggtcatcgtttattaccagctgctgaagctcacgcacgtttaaagacacagttaccacagcagaaattacagttacaggaagcatgcatgcgtaaggaaaagagtgttgctgttttagaacatcagttagttgaagttgaagaaacagttcgtcagttccgtggtgctgttggtgaacagttaggcaagatgcgtgttttcttagctgcattagaaggcagtttagaccgtgaagcagaacgtgttcgtggtgaagcaggtgttgctttacgtcgtgaattaggtagtttaaacagttacttagaacagttacgtcagatggaaaaggttttagaagaagttgctgacaagccgcagaccgaattcttaatgaaatactgcttagttaccagtcgtttacagaagatcttagcagaaagtccaccaccagctcgtttagacatccagttaccaattatctcagatgacttcaaattccaggtttggcgtaagatgttccgtgctttaatgccagctttagaagaattaacctttgacccgagtagtgctcacccgagtttagttgttagtagtagtggccgtcgtgttgaatgcagtgaacagaaggctccgccggctggtgaagacccgcgtcagttcgacaaggctgttgctgttgttgctcaccagcagttaagtgaaggcgaacactactgggaagttgatgttggcgacaagccgcgttgggctttaggcgttatcgctgctgaagctccacgtcgtggtcgtttacacgctgttccaagtcagggcttatggttattaggtttacgtgaaggcaagatcttagaagcacacgttgaagctaaggaaccgcgtgctttacgtagtccagaacgtcgtccaacccgtattggcttatacttaagtttcggcgacggcgttttaagtttctacgatgctagtgacgctgacgctttagttccgttatttgctttccacgaacgtttaccacgtccagtttacccattcttcgacgtttgctggcacgacaagggcaagaatgctcagccgttattattagttggtccagaaggcgctgaagcttaagtcgacgcgt

SEQ ID NO: 6 - protein, 38 aa, Artificial Sequence
  signal peptide SP-prtP cell wall associate protease
MQRKKKGLSILLAGTVALGALAVLPVGEIQAKADTNSD

SEQ ID NO: 7 - DNA, 114 bp, Artificial sequence
  signal peptide SP-prtP; cell wall associated protease
atgcaacgtaaaaagaaaggtttaagtatcttattagctggtacagttgctttaggtgctttagctgttttaccagttggcgaaatccaagcaaaggctgacacaaacagtgat

SEQ ID NO: 8 - protein, 36 aa, Artificial sequence
  signal peptide SP-slpA; SplA, cell surface protein
MKKNLRIVSAAAAALLAVAPVAASAVSVNAAATTTN

SEQ ID NO: 9 - DNA, 108 bp, Artificial sequence
  signal peptide SP-slpA; SplA, cell surface protein
atgaagaaaaatttaagaattgttagcgctgctgctgctgctttattagctgttgctccagttgctgcttcagctgtttctgttaacgctgctgcaaccactaccaac

(19) United States
(12) Patent Application Publication
(10) Pub. No.: US 2022/0273763 A1
(43) Pub. Date: Sep. 1, 2022

(54) Treatment for Inflammatory Bowel Disease and Radiation-induced Intestinal Injury

(71) Applicant: OHIO STATE INNOVATION FOUNDATION, COLUMBUS, OH (US)

(72) Inventors: Jianjie MA, Powell, OH (US); Chunyu ZENG, Chongqing (CN); Yu HAN, Chongqing (CN); Donghai YANG, Chongqing (CN); Zhengfang GONG, Chongqing (CN); Zhongshu ZHOU, Chongqing (CN); Jin ZHONG, Chongqing (CN)

(73) Assignees: OHIO STATE INNOVATION FOUNDATION, COLUMBUS, OH (US); DAPING HOSPITAL, ARMY MEDICAL UNIVERSITY, Chongqing (CN); Institute of Microbiology, Chinese Academy of Sciences, Beijing (CN)

(21) Appl. No.: 17/740,547

(22) Filed: May 10, 2022

Related U.S. Application Data
(63) Continuation of application No. PCT/US2019/060684, filed on Nov. 11, 2019.

(51) Int. Cl. A61K 38/17; A61K 35/747; A61P 1/00; A61K 9/00; A23L 33/00; A23L 33/195; A23L 33/135

(52) U.S. Cl. CPC A61K 38/1709; A61K 35/747; A61P 1/00; A61K 9/0053; A23L 33/40; A23L 33/195; A23L 33/135; A61K 2035/11

(57) ABSTRACT

Compositions for and methods of treating irritable bowel disease are provided. The compositions and methods employ MG53, which can be in the form of recombinant human MG53. The MG53 can be administered in a form to avoid or bypass gastric digestion or degradation. The composition can include a live MG53-expressing microbe, such as a probiotic composition that expresses MG53 in the gastrointestinal tract of a subject. An enteric release composition comprising MG53 is also provided.

### CROSS-REFERENCE TO EARLIER FILED APPLICATIONS

This application claims the benefit of and is a continuation of application No. PCT/US2019/060684 filed Nov. 11, 2019, the entire disclosure of which is hereby incorporated by reference.

### INCORPORATION BY REFERENCE

In compliance with 37 CFR 1.52(e)(5), the instant application contains Sequence Listings which have been submitted in electronic format via EFS and which are hereby incorporated by reference. The sequence information contained in electronic file named TRIM41PCT_ST25.txt, size 13 KB, created on Nov. 1, 2019, using Patent-in 3.5.1, and Checker 4.4.6 is hereby incorporated by reference in its entirety. The information (sequence listing) recorded in said TRIM41PCT_ST25.txt file is identical to that submitted in the priority PCT international application under Rule 13ter.1(a).

### FIELD OF THE INVENTION

The present invention concerns compositions for and methods of treating inflammatory bowel diseases (IBD's) and radiation-induced intestinal injury. An oral composition comprising a therapeutically effective amount of MG53 is administered to a subject in need thereof according to a suitable dosing regimen. The invention also provides a method of reducing the occurrence and/or frequency of inflammatory episodes associated with IBD or mitigation of radiation-induced intestinal injury.

### BACKGROUND OF THE INVENTION

Inflammatory Bowel Disease (IBD) is a chronic relapsing autoimmune disease characterized by alternating active and relapsing periods, divided into Ulcerative Colitis and Crohn's disease. Ulcerative colitis is a chronic non-specific colonic inflammation with diarrhea, abdominal pain, and mucus pus and bloody stools as the main clinical manifestations. Crohn's disease is a chronic granulomatous inflammation with abdominal pain, diarrhea, fistula, anal lesions and varying degrees of systemic symptoms as the main clinical manifestations. In recent years, with the increasing westernization of lifestyle, the prevalence of IBD has gradually increased in China. The pathogenesis of IBD is still not fully understood, resulting in a low control rate of IBD, causing repeated delays in the course of IBD, affecting the quality of life of patients.

Intestinal injury is also the primary toxicity of radiotherapy for pelvic and abdominal tumors, and it is also one of the common acute complications of radiotherapy. At present, there are no effective drugs to prevent intestinal injury in the clinic.

The main pathological changes in inflammatory bowel disease are extensive acute and chronic inflammation of the intestinal mucosa with ulceration, erosion and necrosis, and this pathological change in the intestinal mucosa leads to impaired intestinal epithelial barrier function. Intestinal epithelial cells and intercellular tight junctions and adhesive junctions together constitute the intestinal epithelial barrier, which is the basis of the intestinal epithelial barrier. Under pathological conditions, necrosis of intestinal epithelial cells leads to increased cell permeability, allowing intestinal pathogens, endotoxins and macromolecules to enter the other tissues or circulatory system through the intestinal barrier, inducing the occurrence of immune inflammatory reactions, while the intestine After the inflammation of the tract, it can cause further necrosis of the intestinal epithelial cells, thereby amplifying the inflammatory reaction and forming a vicious circle, which eventually leads to repeated IBD. Repairing necrosis of intestinal epithelial cells is the main research direction for the prevention and treatment of IBD.

Intestinal epithelial cell membrane rupture is the main pathological basis of intestinal epithelial cell necrosis and intestinal epithelial barrier destruction. The cell membrane repair protein MG53 protein is a member of a family of 477 amino acids of the tripartite motif. Its structure contains three domains, from the N-terminus to the C-terminus, the zinc finger domain, the B-box and the coiled-coil structure, specific. MG53 is predominantly expressed in myocardium and skeletal muscle cells. Previous studies have found that after cell membrane damage, MG53 can be transferred from the cytoplasm to the cell membrane, accumulate in the membrane damage, close the membrane gap, and repair the cell membrane. Administration of MG53 in a model of cardio-kidney-lung injury and a mouse model of muscular atrophy can promote cell survival through membrane repair function, and achieve therapeutic effects, but no reports have been reported in repairing intestinal epithelial cells. On the other hand, the E3 ubiquitin ligase Ring finger domain in the MG53 protein structure acts as an E3 ubiquitin ligase, induces ubiquitination of insulin receptor 1 (IRS1), and accelerates the attenuation of IRS1, so that MG53 protein can also act as an important regulator in protein attenuation.

Methods of preparing and/or isolating MG53 are known: U.S. Pat. No. 7,981,866, WO2008/054561, WO2009/073808, US2011/0202033, US2011/0287004, US2011/0287015, US2013/0123340, WO2011/142744, WO2012/061793, U.S. Pat. Nos. 8,420,338, 9,139,630, 9,458,465, 9,494,602, US2014/0024594, WO2012/134478, WO2012/135868, US2015/0110778, WO2013/036610, US2012/0213737, and WO2016/109638.

The technology of the present inventors has been copied by others for use in the treatment of irritable bowel disease and disclosed in Chinese patent application publications No. CN 108339111A, No. CN 108478800A, and CN 109528684A, the entire disclosures of which are hereby incorporated by reference. However, due to the natural specificity of the protein, the mode of administration is limited. Moreover, proteins are known to be degraded in the stomach and digested in the gastrointestinal (GI) tract. MG53 is therefore not simply administered orally. It would be an advancement in the art to provide MG53 in a form suitable for oral (peroral) administration to provide a clinical benefit to tissues of the GI tract.

### SUMMARY OF THE INVENTION

The present inventors seek to treat IBD by oral administration of MG53 or of an MG53-expressing material. The compositions and dosage forms herein can be used to express (release) MG53 in the intestinal tract in a non-antigenic, safe to use, convenient to take, and convenient manner suitable for mass production of MG53 protein. The composition and method herein can be used to treat inflammation of tissue along the entire gastrointestinal tract.

One of the objects of the present invention is to provide the use of MG53 as a marker for diagnosing or treating ulcerative colitis. Another object of the present invention is to provide a recombinant human MG53 protein for preventing and/or treating inflammatory diseases. The invention relates to a drug or a healthcare product for treating enteric diseases. A third object of the present invention is to provide a recombinant lactic acid bacterium that secretes human MG53 protein and the use of said bacterium for preparing a medicament or a healthcare product for use in preventing and treating inflammatory bowel disease. A fourth object of the present invention is use of human MG53 protein in the preparation of a medicament or a healthcare product for repairing intestinal epithelial cell damage. In order to achieve these objectives, the present invention provides the following technical solutions.

An aspect of the invention provides a method of forming MG53 in the gastrointestinal tract of a subject, the method comprising administering to said subject a composition comprising a live (or active) culture of an MG53-expressing microbe. In some embodiments, the method comprises administering exogenous MG53 and administering said composition comprising a live (or active) culture of an MG53-expressing microbe to said subject. In some embodiments, the exogenous MG53 and said MG53-expressing microbe are in the same composition. In some embodiments, the exogenous MG53 and said MG53-expressing microbe are in different compositions. Exogenous MG53 can be administered systemically for IBD or radiation induced injury and can be combined with the MG53-expressing microbe and taken orally. The invention also provides a method of treating and/or preventing IBD comprising administering to a subject an effective amount of said composition(s). The invention also provides a method of reducing the occurrence of and/or reducing the severity of symptoms associated with IBD, the method comprising administering to a subject an effective amount of said composition(s).

The invention also provides a probiotic composition comprising a safe and non-antigenic MG53-expressing microbe that expresses MG53 in the GI tract of a subject. The invention also provides a method of treating and/or preventing IBD comprising administering to a subject an effective amount of said probiotic composition. The invention also provides a method of reducing the occurrence of and/or reducing the severity of symptoms associated with IBD, the method comprising administering to a subject an effective amount of said probiotic composition.

In some embodiments, the probiotic composition comprises a live (or active) culture of an MG53-expressing engineered microbe. The MG53-expressing engineered microbe may comprise live bacteria, live yeast, or a combination thereof. The microbe may be selected from the group consisting of Lactobacillus, Lactococcus, Micrococcus, (e.g. micrococcus acidi lactici), Bifidobacterium, bacteroides, Escherichia coli, Saccharomyces (e.g. Saccharomyces boulardii).

In some embodiments, at least one prebiotic is included in a composition comprising a live (or active) culture of an MG53-expressing engineered microbe. In some embodiments, at least one prebiotic is administered to a subject receiving live (or active) culture of an MG53-expressing engineered microbe. The invention thus provides methods wherein a subject receiving live (or active) culture of an MG53-expressing engineered microbe is also administer at least one prebiotic. The at least one prebiotic can be administered with or separately from the live (or active) culture of an MG53-expressing engineered microbe.

The invention also provides an enteric release composition comprising MG53, at least one enteric release material, and one or more pharmaceutical excipients. The enteric release composition can be used to deliver MG53 to the gastrointestinal tract of a subject. The invention also provides a method of treating and/or preventing IBD comprising administering to a subject an effective amount of said enteric release composition. The invention also provides a method of reducing the occurrence of and/or reducing the severity of symptoms associated with IBD, the method comprising administering to a subject an effective amount of said enteric release composition.

An ulcerative colitis model in mice has been developed. We have determined that serum MG53 may be a potential therapeutic marker for colonic damage in ulcerative colitis. In particular, the content of MG53 in the circulating blood (or serum) of the ulcerative colitis (UC) model mouse is lower than that of normal mice, and the MG53 content in colonic tissue is higher than that of the normal mice. Accordingly, lower than average MG53 content in the circulating blood (or serum) in combination with higher than average MG53 content in colonic tissue is indicative of IBD, such as UC. MG53 can, therefore, be used as a diagnostic biomarker for ulcerative colitis. In some embodiments, the average MG53 content in circulating blood (or serum) is determined from a population of subjects (not having IBD) similar in demographics to the subject being treated. In some embodiments, the average MG53 content in colonic tissue is determined from a population of subjects (not having IBD) similar in demographics to the subject being treated.

One aspect of the invention utilizes the MG53 knockout mouse ulcerative colitis model and demonstrates that MG53 knockout mice are more susceptible to dextran sodium sulfate-induced colitis, UC-related weight loss, UC-related diarrhea, UC-related occult blood, and shortened colon length. To increase the survival rate of mice, or other mammal(s), with IBD, recombinant human MG53 (rhMG53) has a significant effect in the treatment of inflammatory bowel disease, especially in the treatment of ulcerative colitis and Crohn's disease. Therefore, the rhMG53 protein can be used for the preparation of a medicament, a composition, a health supplement, a food or an additive for preventing and/or treating inflammatory bowel disease. In some embodiments, the IBD is selected from the group consisting of ulcerative colitis, and Crohn's disease.

The present inventors have discovered that rhMG53 protein and secreted active rhMG53 nisin protein can inhibit dextran sodium sulfate-induced colitis in mice (as recognized by the experimental animal model of ulcerative colitis disclosed herein) by intramuscular or oral administration, respectively, thereby resulting in reduced weight loss caused by IBD, reduced diarrhea and occult blood caused by IBD, inhibition of the shortening of colon length caused by IBD, and improved the survival rate. The above results indicate that recombinant rhMG53 has a good effect on ulcerative colitis in mammals and can be used for the prevention and treatment of ulcerative colitis.

rhMG53 protein and secreted active rhMG53 nisin can also reduce 2,4,6-trinitrobenzenesulfonic acid-induced colitis (as recognized by the experimental animal model of Crohn's disease), relieve loss of bodyweight, alleviate IBD-associated symptoms, increase the survival rate, and inhibit the shortening of the colon length. The above results indicate that recombinant rhMG53 protein and secreted active rhMG53 nisin have a good effect on mouse Crohn's disease. Therefore, the application of the recombinant lactic acid bacteria secreting human MG53 protein or MG53 nisin in the preparation of a medicament, a composition, a health care product, a food or an additive for preventing and treating inflammatory bowel disease.

The invention also provides a genetically-modified non-natural probiotic microbe comprising an MG53-gene expression vector, said microbe expressing and secreting human MG53 protein. In some embodiments, said microbe is a recombinant lactic acid bacterium. In some embodiments, the expression vector is ligated into the pNZ8148 vector polyclonal cleavage site by the MG53 gene.

The use of recombinant human MG53 protein for the preparation of a medicament or a healthcare product for repairing intestinal epithelial cell damage is also contemplated.

The above recombinant rhMG53 protein can be mixed with a pharmaceutically acceptable carrier for the preparation of various preparations for the prevention and treatment of ulcerative colitis and Crohn's disease. The composition of the invention can be administered orally, sublingually, transdermally, by injection, by instillation, to the mucous membrane, by spray, by infusion, rectally, or parenteral administration.

Another aspect of the invention provides a method of diagnosing IBD, the method comprising: obtaining a sample of serum and a sample of gastrointestinal tissue from a test subject; measuring the content of MG53 in said samples; comparing said content of MG53 to the content of MG53 in the serum and GI tissue of another subject not having IBD; whereby the finding of an elevated (above average) level, as compared to the average level observed in a population of subjects (not having IBD) similar in demographics to the test subject, of MG53 in the colonic tissue of said test subject in combination with a finding of reduced level (below average), as compared to the average level observed in a population of subjects (not having IBD) similar in demographics to the test subject, of MG53 in the serum of said test subject is indicative (diagnostic) of IBD in the test subject.

Another aspect of the invention provides a method of monitoring the progression of IBD in a subject, the method comprising: obtaining a first sample of serum and a first sample of gastrointestinal tissue from a subject; measuring the content of MG53 in said first samples; at a later time point, obtaining a second sample of serum and a second sample of gastrointestinal tissue from a subject; measuring the content of MG53 in said second samples; comparing said content of MG53 in said first samples to the content of MG53 in said second samples. If the content of MG53 in serum is greater in the first sample than in the second sample and the content of MG53 in GI tissue is lesser than the first sample than in the second sample, then the subject's IBD will be deemed to have progressed/advanced.

Embodiments of the invention exclude compositions comprising single unaltered natural product; however, said compositions may comprise mixtures of said unaltered natural product(s) along with other components thereby resulting in manmade compositions not present in nature. Embodiments of the invention exclude processes that employ solely unaltered natural processes; however, said processes may comprise a combination of said unaltered natural processes along with one or more other non-natural steps, thereby resulting in processes not present in nature. Embodiments of the invention may also include new uses (new methods of treatment) for natural products, new compositions comprising said natural products, and new methods employing said natural products.

The invention includes all combinations of the aspects, embodiments and sub-embodiments disclosed herein. Other features, advantages and embodiments of the invention will become apparent to those skilled in the art by the following description, accompanying examples and appended claims.

### BRIEF DESCRIPTION OF THE FIGURES

The following drawings are part of the present specification and are included to further demonstrate certain aspects of the invention. The invention may be better understood by reference to one or more of these drawings in combination with the detailed description of the specific embodiments presented herein.

FIGS. 1A-1D concern the content and localization of MG53 in blood and colon of mice with ulcerative colitis. FIG. 1A depicts Western Blot gels indicating the content of MG53 in the serum of mice with ulcerative colitis. FIG. 1B depicts photographs of the colonic tissue of the mice to demonstrate the tissue localization of colonic MG53 in the mice. FIG. 1C depicts a quantitative Western Blot gel of MG53 and GAPDH in the serum, and FIG. 1D depicts a chart quantifying the level of expression of MG53 relative to the level of expression of GAPDH based upon the gel of FIG. 1C. These analyses were conducted according to Example 18.

FIGS. 2A-2C depict chronological charts of changes in bodyweight (FIG. 2A), disease activity index (FIG. 2B), and survival rate (FIG. 2C) for MG53 knockout mice used in the ulcerative colitis model conducted according to Example 2.

FIGS. 3A-3C depict chronological charts for the effect of MG53 administration upon the changes in bodyweight (FIG. 3A), disease activity index (FIG. 3B), and survival rate (FIG. 3C) for MG53 knockout mice used in the ulcerative colitis model conducted according to Example 3.

FIG. 4A depicts a photograph of sections of the colon for mice treated with MG53 versus untreated mice or DSS mice. FIG. 4B depicts a chart quantifying the length of the colon sections of FIG. 4A. FIG. 4C depicts photographs of tissue sections of the colons of FIG. 4A.

FIG. 4D depicts a chart quantifying the histological/pathological score for the tissue sections of FIG. 4C.

FIG. 5A depicts a chart of the impact of administration of recombinant human MG53 (rhMG53) upon survival rate of mice in the mouse colitis model, whereby colitis was induced by 2,4,6-trinitrobenzenesulfonic acid (TNBS) administration. FIG. 5B depicts a chronological chart of the bodyweight of those mice. Evaluation was conducted according to Example 4.

FIG. 6A depicts a generalized schematic of the vector used to promote secretion of active human MG53 protein. FIG. 6B depicts a photograph of the PCR analysis of the expressed MG53 (which is also referred to as TRIM72). FIG. 6C depicts a photograph of the Western Blot gel of the expressed MG53. Preparation was conducted according to Example 5.

FIG. 7 depicts a chart of the effect of administration of the rhMG53, as expressed in the form of active human MG53 protein, on the survival rate of mice used in the ulcerative colitis model. Evaluation was conducted according to Example 6.

FIG. 8 depicts a chart of the effect of secretion of active human MG53 protein on the survival rate of mice used in the ulcerative colitis model. Evaluation was conducted according to Example 7.

FIG. 9 depicts an in vitro release profile for the MG53-containing enteric release composition of Example 9. The release profile was determined according to Example 10.

FIG. 10 depicts a chart of the change in bodyweight over time for mice having DSS-induced IBD: untreated control group (DSS), rhMG53 treated group (DSS-rhMG53; for the enteric release formulation). The study was conducted according to Example 12. Oral gavage with the enteric release formulation decreased bodyweight loss.

FIG. 11 depicts a chart of the FITC-dextran concentration in the GI tract of the mice of FIG. 10. Oral gavage with the enteric release formulation decreases FITC-dextran levels, thereby indicating improved maintenance of GI integrity.

FIG. 12 depicts a chart of the colon length of the mice of FIG. 10. At ten days post DSS treatment, DSS mice dosed with the MG53 enteric release composition exhibited increased colon length.

FIG. 13 depicts confocal microscopic images of intestinal epithelia of mice with DSS-induced IBD following treatment with FITC encapsulated in EUDRAGIT S-100 or treatment with FITC-rhMG53 encapsulated in EUDRAGIT S-100. Evaluation was conducted according to Example 13.

FIG. 14 depicts a chart of the number of budding enteroids versus the type of treatment for enteroids derived from intestinal stem cells following treatment with DSS. Evaluation was conducted according to Example 14.

FIG. 15 depicts a generalized diagram of the plasmid used to generate engineered lactobacillus probiotic according to Example 15.

FIG. 16 depicts the generalized chromosomal DNA maps of the wild type (WT) and the recombinant strain (M1) of Example 15.

FIG. 17 depicts Western Blot gels for standardized solutions containing MG53 (left gel) and for lactobacillus-derived solutions containing MG53 (right gel).

FIG. 18 depicts a chart of bodyweight versus time for mice with DSS-induced IBD: control group; lactobacillus-treated group (DSS+Lacto Co); and engineered MG53-secreting lactobacillus-treated group (DSS+Lacto MG53). Evaluation was conducted according to Example 16. The reduction in bodyweight loss by treatment with engineered MG53-secreting lactobacillus evidences its efficacy.

FIG. 19 depicts a chart of FITC-dextran concentration in the serum of the mice of FIG. 18. FITC-dextran level in the serum is reduced by the engineered lactobacillus, indicating an improved intestine barrier integrity.

FIG. 20 depicts a chart of the colon length of the mice of Example 17: control (no irradiation); IR (irradiated); IR+MG53 (irradiated and MG53 treatment).

FIG. 21 depicts a chart of FITC-dextran concentration in the serum of the mice of FIG. 20. FITC-dextran level in the serum is reduced by MG53 treatment, indicating an improved intestine barrier integrity.

FIG. 22 depicts the DNA sequence (SEQ ID NO. 5) for the expression module of MG53 as depicted in FIG. 15. The initial black bases indicate the restriction enzyme digestion site coding sequence. The blue bases indicate the signal peptide (SP-prtP, cell wall-associated protease) coding sequence. The red bases indicate the human MG53 protein coding sequence. The terminal underlined black bases indicate the terminator coding sequence.

FIG. 23 depicts the DNA sequence (SEQ ID NO. 4) for the SP-MG53-6×His in the pNZ8148 plasmid according to Example 5. The initial underlined blue bases indicate secretory signal peptide Usp45 coding sequence in Lactococcus lactis. The black bases indicate the human MG53 coding sequence. The underlined bold red bases indicate the 6×His tag coding sequence. The single-underlined green bases indicate the repA gene coding sequence. The double-underlined green bases indicate the CmR, chloramphenicol acetyltransferase coding sequence The remaining green bases indicate the pNZ8148 gene coding sequence.

FIG. 24 depicts the plasmid restriction map (4649 total base pairs; SEQ ID NO. 4) for the SP-MG53-6×His in the pNZ8148 plasmid.

FIG. 25 depicts the DNA sequence (SEQ ID NO. 1) for the expression module of MG53 as depicted in FIG. 24. The blue bases indicate the secretory signal peptide of Usp45 protein precursor cDNA coding sequence. The black bases indicate the human MG53 protein coding sequence. The red bases indicate the 6×His coding sequence.

### DETAILED DESCRIPTION OF THE INVENTION

Hereinafter, preferred embodiments of the present invention will be described in detail with reference to the accompanying drawings.

MG53 protein (also referred to as mitsugumin 53 or TRIM72) is known in the art. Unless specified otherwise, all embodiments of the invention comprising or employing “MG53” include all known forms of MG53. It also refers to recombinant human MG53 (rhMG53). As used herein and unless otherwise specified, the term MG53 (or MG53 protein) refers to the MG53 protein present as the native form, optimized form thereof, mutant thereof, derivative thereof or a combination of any two or more of said forms. Native MG53 contains 477 amino acids that are well conserved in different animal species. Methods of preparing and/or isolating MG53 are known: U.S. Pat. No. 7,981,866, WO2008/054561, WO2009/073808, US2011/0202033, US2011/0287004, US2011/0287015, US2013/0123340, WO2011/142744, WO2012/061793, U.S. Pat. Nos. 8,420,338, 9,139,630, 9,458,465, 9,494,602, US2014/0024594, WO2012/134478, WO2012/135868, US2015/0110778, WO2013/036610, US2012/0213737, WO2016/109638, the entire disclosures of which, including sequence information therein, are hereby incorporated by reference.

The sequence listing information for native MG53, and variants or various forms thereof, is disclosed in U.S. Pat. Nos. 7,981,866 and 9,139,630, the entire disclosures of which, including sequence information therein, are hereby incorporated by reference. The sequence listing information for a cDNA that encodes optimized native human MG53, or a fragment thereof, is disclosed in U.S. Pat. No. 9,139,630, the entire disclosure of which, including sequence information therein, is hereby incorporated by reference.

As used herein in reference to MG53, the term “mutant” means a recombinant form of MG53 having an amino acid change (replacement) of one, two, three or more amino acids in the amino acid sequence of native MG53. Mutant forms of MG53 and methods of preparing the same are known: US2015/0361146, EP3118317, WO2015/131728, U.S. Pat. No. 9,139,630, the entire disclosures of which, including sequence information therein, are hereby incorporated by reference.

As used herein the term “endogenous MG53”, refers to MG53 present in a subject prior to treatment with a composition or method according to the invention. As used herein, exogenous MG53 is nonendogenous MG53.

As used herein, nisin refers to a polycyclic antibacterial peptide produced by the bacterium Lactococcus lactis. It has 34 amino acid residues, including the uncommon amino acids lanthionine (Lan), methyllanthionine (MeLan), didehydroalanine (Dha), and didehydroaminobutyric acid (Dhb).

As used herein, the term “prebiotic” refers to a material in food that induces the growth or activity of beneficial microorganism such as bacteria and fungi. A prebiotic can be found in food or can be added to a composition of the invention. Dietary prebiotics are typically nondigestible fiber compounds that pass undigested through the upper part of the GI and stimulate the growth or activity of advantageous bacteria that colonize the large bowel (colon) by acting as a substrate for them. Prebiotics typically include plant derived carbohydrates, fructans, galactans, fructooligosaccharides, inulins, galactooligosaccharides, resistant starch, pectin, beta-glucans, xylooligosaccharides.

The invention relates to medicinal use of the protein MG53. Recombinant human MG53 secreted by micrococcus acidi lactici is used for the preparation of drugs, pharmaceutical compositions, foods, healthy products, food additives and the like for preventing and/or treating inflammatory bowel diseases. The recombinant human MG53 has excellent preventive (prophylactic) and therapeutic effect for treatment of IBD, including ulcerative colitis or Crohn disease. The composition of the invention is free of toxicity or side effects and can be durably and effectively applied to the preparation of drugs, pharmaceutical compositions, foods, healthy products or food additives for preventing and/or treating the inflammatory bowel diseases. The compositions, foods, healthy products or food additives described herein can be used for preventing and treating the inflammatory bowel diseases to provide a substantial clinical benefit.

The present inventors have unexpectedly discovered that administration of MG53 to the gastrointestinal tract or administration of a MG53-expressing material to the gastrointestinal tract is effective for the treatment of irritable bowel disease (IBD) and inflammatory symptoms associated with IBD. As used herein, IBD is intended to include, by way of example and without limitation, any disease or disorder causing inflammation of gastrointestinal tissue. In some embodiments, said inflammation includes inflammation of any GI tissue from the mouth to the anus. In particular embodiments, the tissue is the small intestine, colon, and/or rectum. More particularly, IBD includes ulcerative colitis and Crohn's disease.

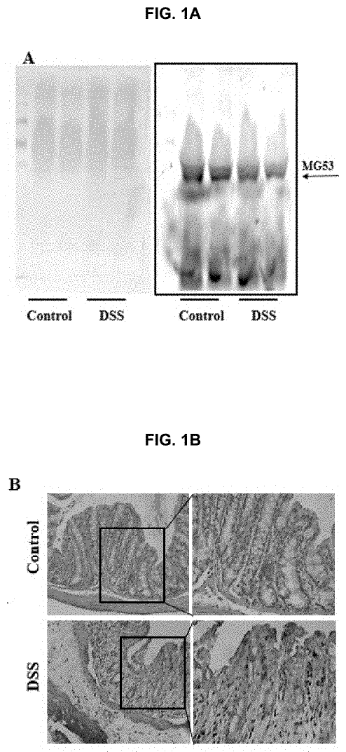

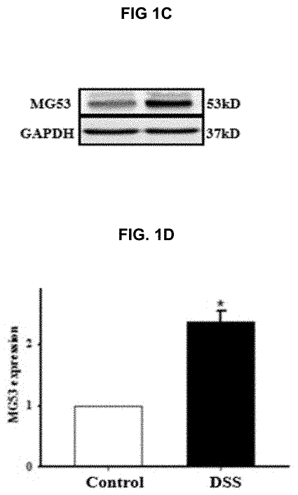

A mouse model for ulcerative colitis was developed (Example 1) for use in evaluating the effect of MG53 upon the condition. After being treated with DSS to induce IBD, MG53 knockout mice exhibit reduced MG53 content in blood/serum (FIG. 1A) and elevated MG53 content in colonic tissue (FIG. 1B).

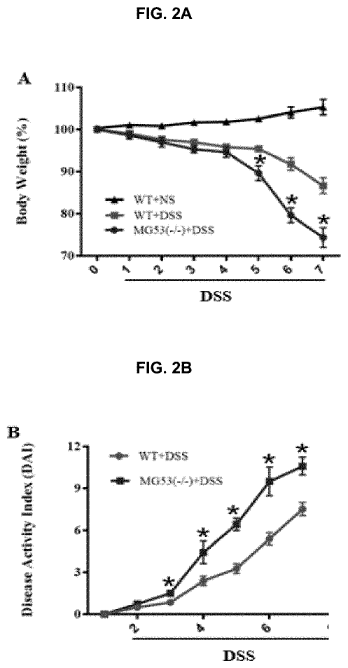

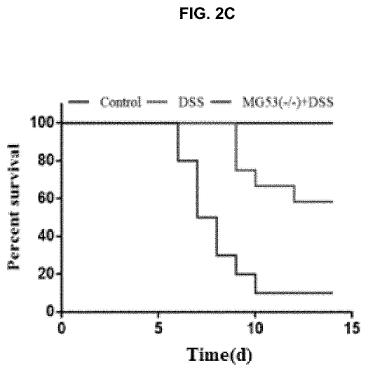

Wild-type and MG53 knockout mice were evaluated for bodyweight loss, disease activity index and survival rate after being treated with DSS to induce IBD. It was determined that WT mice exhibit reduced bodyweight loss (FIG. 2A), reduced disease activity index (FIG. 2B), and better survival rate (FIG. 2C).

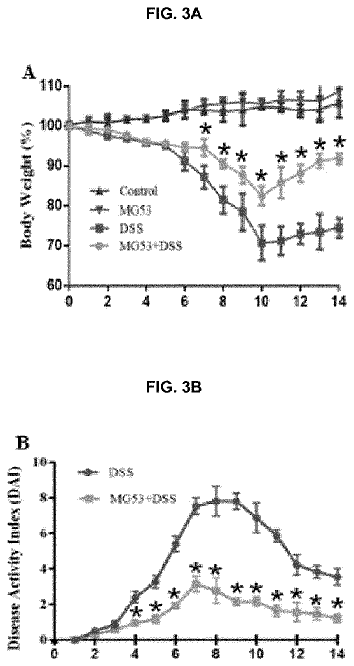

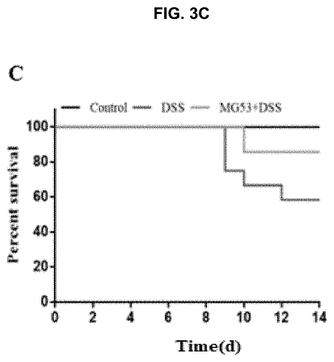

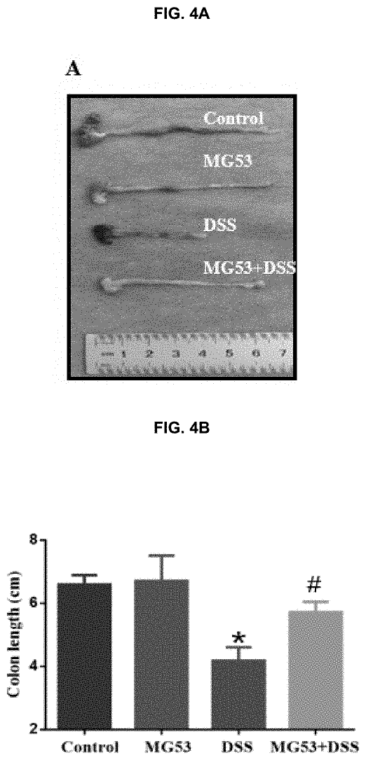

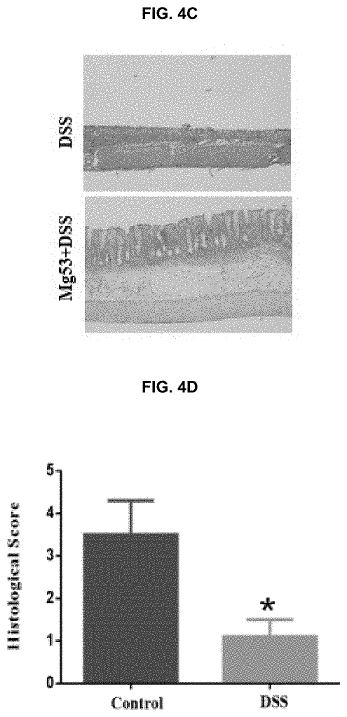

The impact of MG53 treatment upon DSS-induced IBD was evaluated in C57 mice. Regarding bodyweight loss (FIG. 3A), MG53 provided slightly reduced body weight loss, meaning slightly increased bodyweight for the control mice. However, in DSS-treated mice, MG53 substantially decreased bodyweight loss caused by DSS. Regarding disease activity index (FIG. 3B), MG53 substantially improved the disease activity index in DSS-treated mice. MG53 also increased the survival rate for DSS-treated mice (FIG. 3C). The colon length for control mice and MG53-treated (but not DSS-treated) mice was about the same, but MG53 substantially improved colon length for DSS-treated mice (FIGS. 4A and 4B). Moreover, MG53 improved epithelial crypt structure (FIG. 4C).

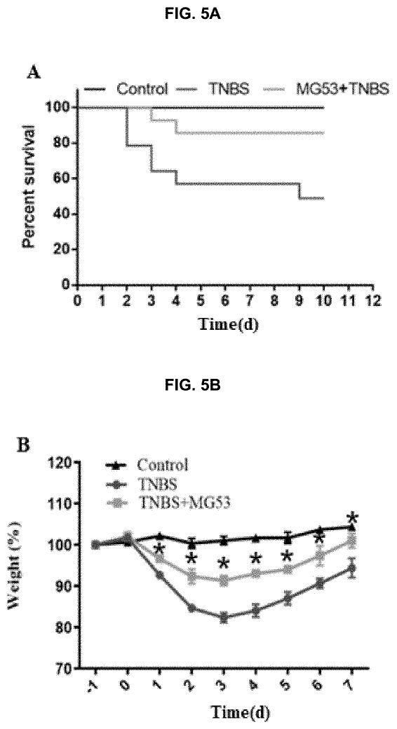

For the Crohn's disease model (Example 4), BALB/c mice were treated TNBS to induce symptoms similar to Crohn's disease. The TNBS-treated mice were then treated MG53, which resulted in improved survival rate (FIG. 5A) and substantially reduced bodyweight loss (FIG. 5B).

Based upon the above in vivo findings, the inventors have determined that MG53 is therapeutically effective at treating IBD characterized as either ulcerative colitis or Crohn's disease when it is administered to a mammal. Systemic or local modes of administration are suitable.

Since MG53 can be degraded by proteases in the GI tract or by the acidic conditions of the stomach, we invented a probiotic composition whereby a safe microbe is engineered to express MG53. The probiotic composition is then administered orally (perorally) to a subject such that the microbe expresses MG53 in the GI tract of a subject.

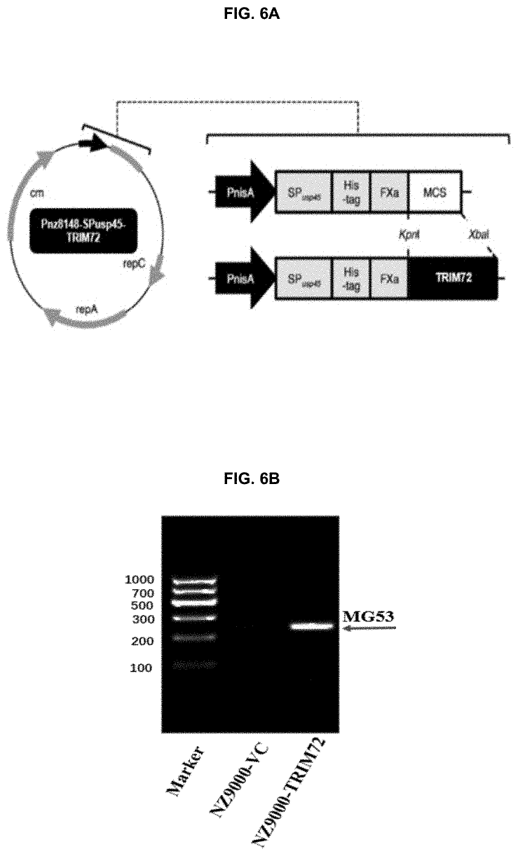

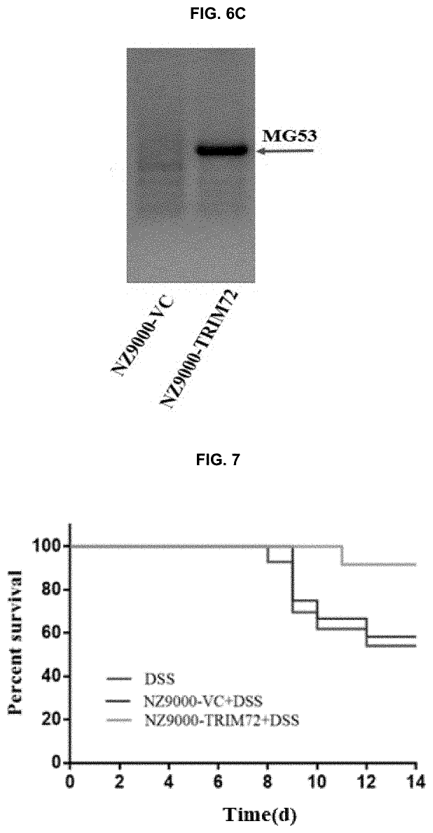

Lactobacillus bacteria was genetically engineered as described in Example 5 to include a plasmid that induces expression of MG53. A food-grade expression vector pNZ8148 was ligated to the MG53 gene, wherein the ligation site is depicted in FIG. 6A. MG53 expression was confirmed by gel (FIGS. 6B and 6C).

For the ulcerative colitis model (Example 6), C57 mice were divided into four groups according to what they were administered: water (control); DSS (DSS group); normal lactobacillus (which does not express MG53; NZ9000−VC+DSS); and engineered lactobacillus that expresses and secretes MG53 (NZ9000−TRIM72+DSS). Administration of the engineered lactobacillus substantially improved survival rate in mice that had also been treated with DSS (FIG. 7).

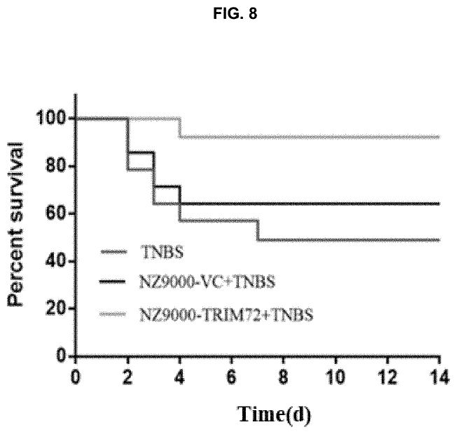

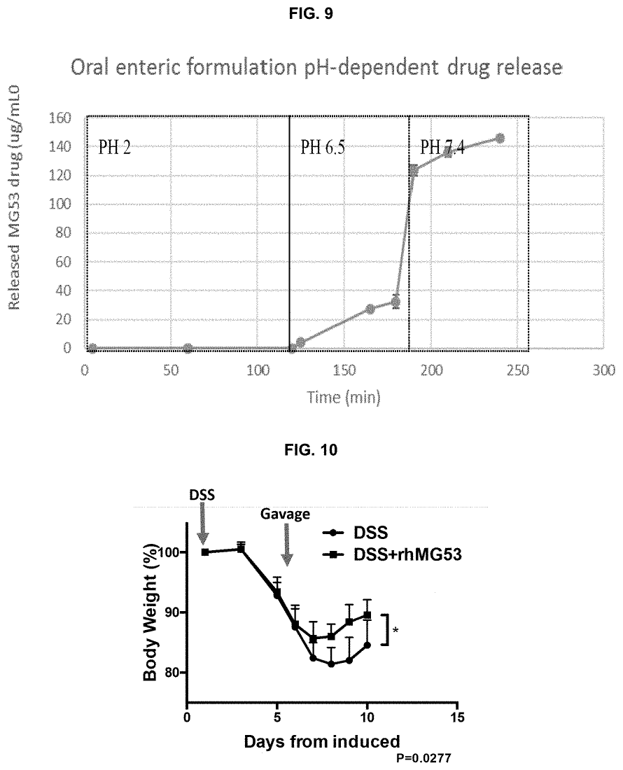

For the Crohn's disease model (Example 7), C57 mice were divided into four groups according to what they were administered: water (control); TNBS (TNBS group); normal lactobacillus (which does not express MG53; NZ9000−VC+TNBS); and engineered lactobacillus that expresses and secretes MG53 (NZ9000−TRIM72+TNBS). Administration of the engineered lactobacillus substantially improved survival rate in mice that had also been treated with TNBS (FIG. 8).

The above in vivo data evidences the efficacy of the probiotic composition of the invention. The invention thus provides a therapeutically effective probiotic composition comprising a genetically engineered microbe that expresses and secretes MG53 and use of said probiotic composition for treating IBD following oral administration of said probiotic composition.

As another means of providing MG53 to the intestinal tract down stream of the stomach, an enteric release (ER) composition comprising MG53 was developed (Examples 8 and 9). The ER composition comprises MG53, an enteric release pharmaceutical excipient, and a cyclodextrin. In particular embodiments, the ER composition comprises MG53, at least one enteric release polymer, and at least one cyclodextrin derivative.

In particular embodiments, the enteric release polymer is a copolymer of methacrylic acid and methyl methacrylate. In particular embodiments, the enteric release polymer has a dissolution pH of ≥about 5, ≥about 5.5, ≥about 6, ≥about 6.5, or ≥about 7.

In particular embodiments, the cyclodextrin derivative is water soluble. In particular embodiments, the cyclodextrin derivative is hydroxypropyl-beta-cyclodextrin.

The MG53 release profile for the ER composition was determined according to Example 10. The ER composition exhibits a typical ER profile. MG53 release began at a pH above 6, above 6.5, above 7 or above 7.5.

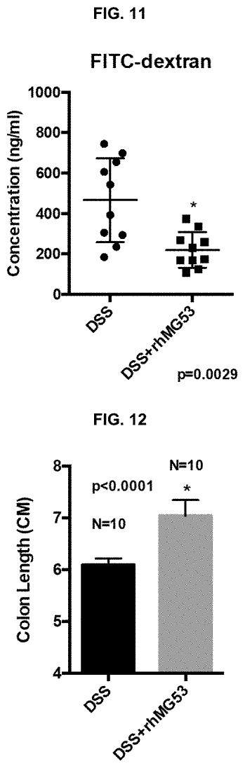

The therapeutic efficacy of the ER composition was evaluated in the ulcerative colitis model according to Example 12. The data indicate that MG53-containing ER composition provides reduced bodyweight loss (FIG. 10), reduced serum levels of FITC-dextran (FIG. 11, thus improved colonic tissue pathology), and increased colon length (FIG. 12) is DSS treated mice.

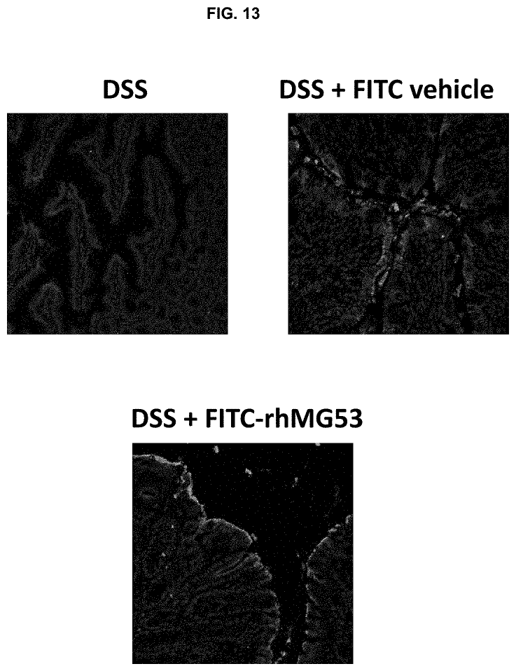

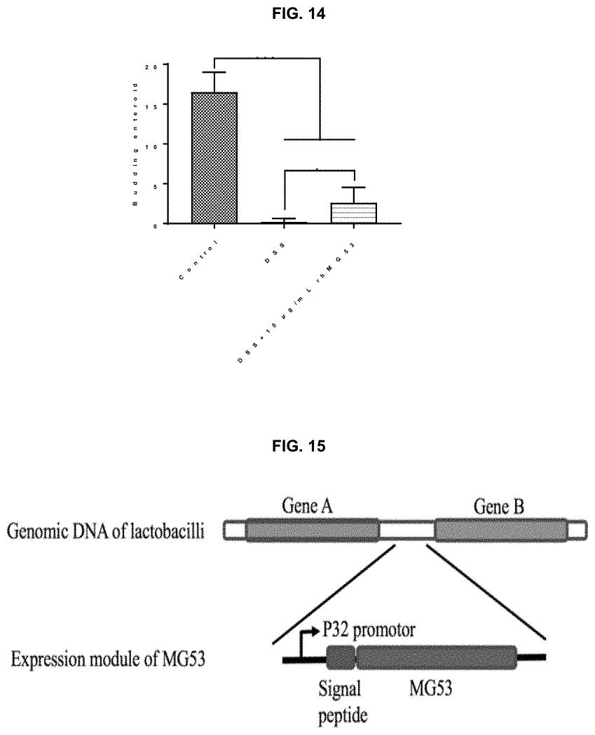

In order to determine a potential mechanism for its, MG53 was fluorescence-labeled with FITC (FITC-MG53) according to Example 13. DSS-treated mice were administered the FITC-MG53 or FITC label (with no protein attached). The data (FIG. 13) demonstrate targeting of FITC-MG53 to the injured intestinal epithelia layer.

Another aspect of the protective action of MG53 was elucidated (Example 140. Enteroids formed from primary stem cells derived from C57BL6/J mice were treated with DSS, which led to the reduction of budding enteroids. When DSS-treated enteroids were then treated with MG53, there was a significant increase in the number of budding enteriods indicating the ability of MG53 to protect stem cells from DSS-induced damage.

Accordingly, the invention provides a method of protecting intestinal stem cells, the method comprising administering an effective amount of MG53.

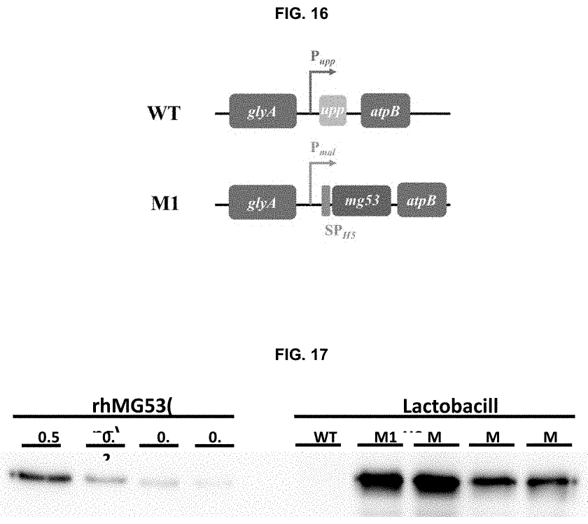

Another probiotic composition according to the invention was prepared according to Example 15. The respective expression module of MG53 was inserted into the genomic DNA of lactobacilli (FIG. 15). The chromosomal DNA maps of the wild type (WT) and the recombinant strain (M1) are depicted in FIG. 16. Secretion of MG53 from the engineered Lactobacillus was confirmed by Western blot (FIG. 17). It can be seen that the WT lactobacillus does not express MG53; however, the engineered lactobacillus (M1) expresses and secretes a substantial amount of MG53. The yield of MG53 was about 1 ng of protein/2.42×10\hat{ }9 CFU of lactobacilli.

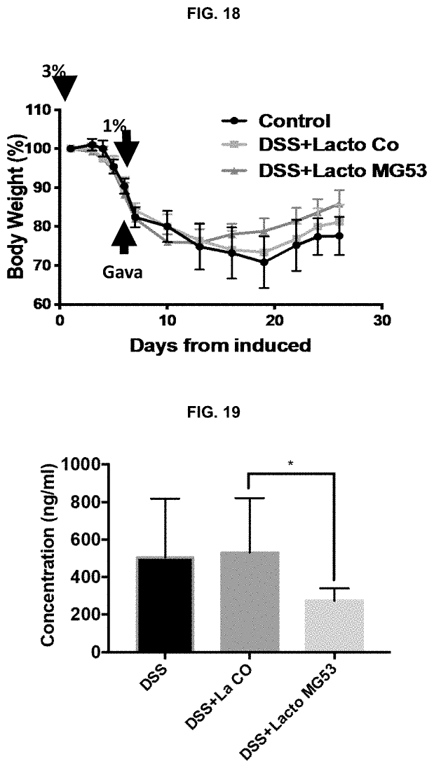

The engineered lactobacillus was evaluated in the ulcerative colitis model using DSS-treated mice according to Example 16. The data (FIGS. 18 and 19) demonstrate efficacy of the engineered lactobacillus. As compared to control, the native Lactobacillus provided insignificant reduction in weight loss (FIG. 18) and insignificant changes in FITC-dextran level in serum (FIG. 19). As compared to control, the engineered Lactobacillus provided significant reduction in weight loss (FIG. 18) and significant decrease in FITC-dextran level in serum (FIG. 19).

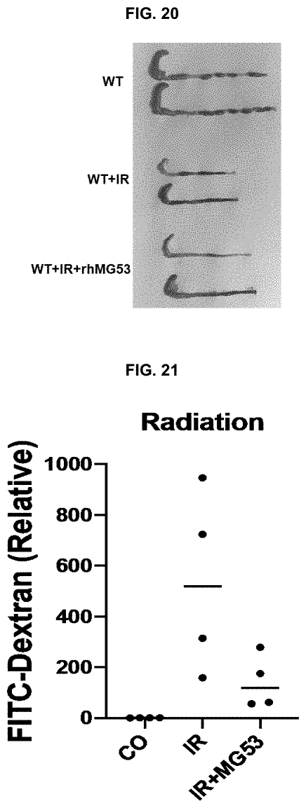

The ability of MG53 to protect against other forms of damage to colonic tissue was further evaluated according to Example 17. Mice were treated with saline or MG53 in saline (tail vein injection) and irradiated with X-rays. The data indicate that irradiation caused shortening of the colon, which was partially mitigated by rhMG53 treatment (FIG. 20), and mice receiving rhMG53 displayed reduced FITC-dextran in the serum, demonstrating the improved integrity of the GI in mice following irradiation (FIG. 21).

The amount of probiotic administered to a subject may vary widely. In general, the probiotic composition (MG53-expressing microbe) will secrete at least about 1 ng of MG53/2×10⁸ CFU of microbe to about 1 ng of MG53/2×10⁹ CFU of microbe.

Suitable concentrations of MG53 in a dosage form include at least 1 ng of MG53/ml, at least 5 ng of MG53/ml, at least 10 ng of MG53/ml, at least 25 ng of MG53/ml, at least 50 ng of MG53/ml, at least 75 ng of MG53/ml, at least 100 ng of MG53/ml, at least 250 ng of MG53/ml, at least 500 ng of MG53/ml, at least 750 ng of MG53/ml, at least 1 μg of MG53/ml, at least 5 μg of MG53/ml, at least 10 μg of MG53/ml, at least 15 μg of MG53/ml, at least 20 μg of MG53/ml, at least 25 μg of MG53/ml, at least 30 μg of MG53/ml, at least 50 μg of MG53/ml, or at least 100 μg of MG53/ml. Higher concentrations are also acceptable, particularly in view the efficacy dose-response trend observed for MG53. These doses can be administered on a frequency as described herein or as determined to be most effective.

Suitable doses of MG53 that can be administered to a subject in one or more dosage forms include at least 1 ng of MG53, at least 5 ng of MG53, at least 10 ng of MG53, at least 25 ng of MG53, at least 50 ng of MG53, at least 75 ng of MG53, at least 100 ng of MG53, at least 250 ng of MG53, at least 500 ng of MG53, at least 750 ng of MG53, at least 1 μg of MG53, at least 5 μg of MG53, at least 10 μg of MG53, at least 15 μg of MG53, at least 20 μg of MG53, at least 25 μg of MG53, at least 30 μg of MG53, at least 50 μg of MG53, or at least 100 μg of MG53. Such doses can be on a total body weight basis or a per kg of body weight basis.

The dose of exogenous MG53 can be as low as about 1 up to about 1000 microg per kg of body weight.

The amount of therapeutic compound (MG53) incorporated in each dosage form will be at least one or more unit doses and can be selected according to known principles of pharmacy. An effective amount of therapeutic compound is specifically contemplated. By the term “effective amount”, it is understood that, with respect to, for example, pharmaceuticals, a pharmaceutically (therapeutically) effective amount is contemplated. A pharmaceutically effective amount is the amount or quantity of a drug or pharmaceutically active substance which is sufficient to elicit the required or desired therapeutic response, or in other words, the amount which is sufficient to elicit an appreciable biological response when administered to a patient.

The term “unit dosage form” is used herein to mean a dosage form containing a quantity of the drug, said quantity being such that one or more predetermined units may be provided as a single therapeutic administration.

The dosage form is independently selected at each occurrence from the group consisting of liquid solution, suspension, tablet, capsule, sachet or powder.

Compositions and dosage forms of the invention can further comprise one or more pharmaceutically acceptable excipients. Dosage forms can comprise one or more excipients independently selected at each occurrence from the group consisting of acidic agent, alkaline agent, buffer, tonicity modifier, osmotic agent, water soluble polymer, water-swellable polymer, thickening agent, complexing agent, chelating agent, penetration enhancer. Suitable excipients include U.S.F.D.A. inactive ingredients approved for use in parenteral or oral formulations (dosage forms), such as those listed in the U.S.F.D.A.'s “Inactive Ingredients Database (available on the following website: www.fda.gov/Drugs/InformationOnDrugs/ucm113978.htm; October 2018), the entire disclosure of which is hereby incorporated by reference.

As used herein, an acidic agent is a compound or combination of compounds that comprises an acidic moiety. Exemplary acidic agents include organic acid, inorganic acid, mineral acid and a combination thereof. Exemplary acids include hydrochloric acid, hydrobromic acid, sulfuric acid, sulfonic acid, sulfamic acid, phosphoric acid, or nitric acid or others known to those of ordinary skill; and the salts prepared from organic acids such as amino acids, acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid, methanesulfonic acid, ethane disulfonic acid, oxalic acid, isethionic acid, others acids known to those of ordinary skill in the art, or combinations thereof.

As used herein, an alkaline agent is a compound or combination of compounds that comprises an alkaline moiety. Exemplary alkaline agents include primary amine, secondary amine, tertiary amine, quaternary amine, hydroxide, alkoxide, and a combination thereof. Exemplary alkaline agents include ammonia solution, ammonium carbonate, diethanolamine, monoethanolamine, potassium hydroxide, sodium borate, sodium carbonate, sodium bicarbonate, sodium hydroxide, triethanolamine, diethanolamine, monobasic phosphate salt, dibasic phosphate salt, organic amine base, alkaline amino acids and trolamine, others known to those of ordinary skill in the art, or combinations thereof.

Exemplary excipients (inactive ingredients as defined by the U.S.F.D.A.) that can be included in dosage forms of the invention include, by way of example and without limitation, water, benzalkonium chloride, glycerin, sodium hydroxide, hydrochloric acid, boric acid, hydroxyalkylphosphonate, sodium alginate, sodium borate, edetate disodium, propylene glycol, polysorbate 80, citrate, sodium chloride, polyvinylalcohol, povidone, copovidone, carboxymethylcellulose sodium, Dextrose, Dibasic Sodium Phosphate, Monobasic Sodium Phosphate, Potassium Chloride, Sodium Bicarbonate, Sodium Citrate, Calcium Chloride, Magnesium Chloride, stabilized oxychloro complex, Calcium Chloride Dihydrate, Erythritol, Levocarnitine, Magnesium Chloride Hexahydrate, Sodium Borate Decahydrate, Sodium Citrate Dihydrate, Sodium Lactate, Sodium Phosphate (Mono- and Dibasic-), Polyethylene Glycol 400, Hydroxypropyl Guar, Polyquaternium-1, Zinc Chloride, white petrolatum, mineral oil, hyaluronic acid, artificial tear, or combinations thereof.

One or more antioxidants can be included in a composition of dosage form of the invention. Exemplary antioxidants include SS-31, NAC, glutathione, selenium, vitamin A, vitamin C, vitamin E, co-enzyme Q10, resveratrol, other GRAS antioxidant, or a combination of two or more thereof.

One or more zinc salts can be included in a composition or dosage form of the invention. Such zinc salt(s) may also be administered to a subject receiving exogenous MG53 or expressed MG53. Pharmaceutically acceptable zinc salts include Zinc gluconate, Zinc acetate, Zinc sulfate, Zinc picolinate, Zinc orotate, Zinc citrate, and other such salts comprising a zinc cation and organic or inorganic anion(s).

It should be understood, that compounds used in the art of pharmaceutical formulations generally serve a variety of functions or purposes. Thus, if a compound named herein is mentioned only once or is used to define more than one term herein, its purpose or function should not be construed as being limited solely to that named purpose(s) or function(s).

As used herein, “pharmaceutically acceptable salts” refer to derivatives of the disclosed compounds wherein the compound is modified by making an acid or base salt thereof. Examples of pharmaceutically acceptable salts include, but are not limited to, mineral or organic acid salts of basic residues such as amines; alkali or organic salts of acidic residues such as carboxylic acids; and others known to those of ordinary skill. The pharmaceutically acceptable salts can be synthesized from the parent therapeutic compound which contains a basic or acidic moiety by conventional chemical methods. Lists of suitable salts are found in Remington's Pharmaceutical Sciences, 17th ed., Mack Publishing Company, Easton, Pa., 1985, p. 1418, the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

MG53 can be used in cotherapy or adjunctive therapy with one or more other active ingredients to treat IBD. Exemplary suitable active ingredients include, among others, U.S.F.D.A. approved drugs for oral (peroral) dosage forms.

Other active ingredients that can be used in cotherapy or adjunctive therapy with MG53 include, by way of example and without limitation, antibiotics (ciprofloxacin (Cipro) and metronidazole (Flagyl), aminosalicylates (5-ASAs), corticosteroids (steroids), immune modifiers (immunomodulators), biologic therapies (biologics), Anti-inflammatories include corticosteroids and aminosalicylates, such as mesalamine (Asacol HD, Delzicol, others), balsalazide (Colazal) and olsalazine (Dipentum), Anti-diarrheal medications, Pain relievers, iron, calcium and vitamin D supplement or combinations thereof.

The therapeutically acceptable dose, maximum tolerated dose (MTD), and minimally effective dose (MED) for each of said active ingredients is well known and set forth in the respective U.S.F.D.A. approved product package insert for each said active ingredients.

A composition, dosage form or formulation of the invention can include one, two or more active ingredients in combination with MG53. The dose of each said active ingredient in said composition, dosage form or formulation of the invention will be a therapeutically effective dose including and above the MED and including and below the MTD.

In some embodiments, the combination treatment of MG53 with another active ingredient provides at least additive therapeutic efficacy. In some embodiments, said combination provides synergistic therapeutic efficacy. In some embodiments, MG53 reduces the occurrence of, reduces the level of, or eliminates adverse events caused by the other active ingredient.

The acceptable concentrations of said excipients are well known in the art and specific concentrations (amounts) thereof are set forth in the package insert or package label of known commercial products containing the same.

It should be understood, that compounds used in the art of pharmaceutics may serve a variety of functions or purposes. Thus, if a compound named herein is mentioned only once or is used to define more than one term herein, its purpose or function should not be construed as being limited solely to that named purpose(s) or function(s).

In the examples below, ranges are specified for the amount of each ingredient. Ranges including “0” as the lowest value indicate an optional ingredient. The lower limit “>0” indicates the respective material is present.

As used herein, the terms “about” or “approximately” are taken to mean a variation or standard deviation of ±10%, ±5%, or ±1% of a specified value. For example, about 20 mg is taken to mean 20 mg±10%, which is equivalent to 18-22 mg.

As used herein, the term “prodrug” is taken to mean a compound that, after administration, is converted within a subject's body, e.g. by metabolism, hydrolysis, or biodegradation, into a pharmacologically active drug. The prodrug may be pharmacologically active or inactive. For example, a prodrug of MG53 (native or mutant) would be converted to the native form or mutant form, respectively, of MG53. The term “precursor” may also be used instead of the term “prodrug”.

As used herein, the term “derivative” is taken to mean: a) a chemical substance that is related structurally to a first chemical substance and theoretically derivable from it; b) a compound that is formed from a similar first compound or a compound that can be imagined to arise from another first compound, if one atom of the first compound is replaced with another atom or group of atoms; c) a compound derived or obtained from a parent compound and containing essential elements of the parent compound; or d) a chemical compound that may be produced from first compound of similar structure in one or more steps. For example, a derivative may include a deuterated form, oxidized form, dehydrated, unsaturated, polymer conjugated or glycosilated form thereof or may include an ester, amide, lactone, homolog, ether, thioether, cyano, amino, alkylamino, sulfhydryl, heterocyclic, heterocyclic ring-fused, polymerized, pegylated, benzylidenyl, triazolyl, piperazinyl or deuterated form thereof.

In the examples below, ranges are specified for the amount of each ingredient. Ranges including “0” as the lowest value indicate an optional ingredient. Compositions with quantities of ingredients falling within the compositional ranges specified herein were made. Compositions of the invention comprising quantities of ingredients falling within the compositional ranges specified herein operate as intended and as claimed.

In view of the above description and the examples below, one of ordinary skill in the art will be able to practice the invention as claimed without undue experimentation. The foregoing will be better understood with reference to the following examples that detail certain procedures for the preparation and use of compositions according to the present invention. All references made to these examples are for the purposes of illustration. The following examples should not be considered exhaustive, but merely illustrative of only a few of the many embodiments contemplated by the present invention. The methods described herein can be followed to prepare and use compositions of the invention and to practice methods of the invention.

MG53 was kindly provided by TRIM-edicine, Inc. (1275 Kinnear RD, Columbus Ohio 43212-1155, U.S.A.).

### Example 1

### Preparation of an Ulcerative Colitis Model

All the animals in the experiment were provided with male C57BL/6 mice by the Experimental Animal Center of Daping Hospital of the Military Medical University, weighing 22-25 g. The surgical operation during the experiment was in accordance with the regulations of the experimental animals of the Army Military Medical University. Mice were randomly divided into the control group and the model group. The control group was given normal sterilized water. The model group was free to drink 4% dextran sulfate sodium (DSS) for 7 days, and the changes in the body signs were observed. On the 8th day of modeling, blood was collected from the eyelids of each group, and the supernatant serum was collected by centrifugation at 5000 rpm for 10 minutes. The colon-extracted tissue sample protein was preserved at 80 degrees and fixed in 4% paraformaldehyde. The protein samples were routinely extracted and quantified by paraffin, embedded in paraffin, and used for the detection of MG53 content and localization in tissue samples. The detection method of serum MG53 content was disclosed (Chinese patent application publication No. CN103430023A). The results are depicted in FIG. 1. The content of MG53 in the circulating blood of mice in the ulcerative colitis model group was lower than that in normal mice. The MG53 content in colon tissue of DSS treated mice was higher than that in normal mice, and MG53 was enriched in intestinal epithelial cells for the DSS treated mice.

### Example 2

### Preparation of MG53 Knockout Mouse Ulcerative Colitis Model

The MG53 knockout mice (kindly provided by Professor Ma Jianjie of TRIM-edicine, Inc.) from the SPF level of the Experimental Animal Center of Daping Hospital of the Army Military Medical University weighed 22 to 25 g. The experiment was conducted in accordance with the regulations of the experimental animals of the Army Military Medical University. Mice were randomly divided into a normal group, a model group (4% DSS), and a MG53 knock-out mouse model group (4% DSS). Except for the normal control group, the other two groups were free to drink 4% dextran sulfate sodium (DSS) for 7 days and then switched to mono-distilled water and continued to drink freely for 7 days. The body weight, disease activity index, and survival rate were continuously observed.

The disease activity index of mice with ulcerative colitis model accurately quantifies the severity of inflammatory bowel disease by disease activity index (DAI). The score is: body mass index (weight is 0, drop 1%-5% is 1 point, decrease 5%-10% is 2 points, 10%-15% is 3 points, the drop is greater than 15% is 4 points), stool trait score (normal 0, loose 2 points, diarrhea 4 points) and stool bleeding score (Normal 0 points, 2 points for recessive bleeding, and 4 points for dominant bleeding). DAI score=(body mass index+stool trait score+stool bleeding score)

The results of MG53 knockout mice with ulcerative colitis model body weight, disease activity index, and survival rate are shown in FIGS. 2A-2C. The model group mice showed a significant increase in disease activity index from day 3, weight loss, diarrhea, and gradually increased as the trial continued, fecal occult blood and blood in the stool, and death began on the 9th day, 14 days. The mortality rate was 50%. The MG53 knock-out mouse ulcerative colitis model mice showed a significant increase in disease activity index from day 2, compared with the model group, the disease activity index, body weight, diarrhea, fecal occult blood and blood in the stool were aggravated; and the survival rate was such that on the 6^{th} day, mice began to die and by the 10^{th} day the mortality rate was 90%.

### Example 3

### Determination of the Protective Effect of rhMG53 Against DSS-Induced Ulcerative Colitis in Mouse Model

C57 mice, maintained in (Specific Pathogen Free) SPF animal facility at Experimental Animal Center of Daping Hospital of the Army Military Medical University were 22 to 25 g in weight. The surgical operation during the experiment was conducted in accordance with the regulations of the Army Military Medical University concerning experimental animals. Mice were randomly divided into three groups: normal group, model group (2.5% DSS), and rhMG53 treatment group. Except for the normal control group, the other 2 groups were free to drink 4% dextran sulfate sodium (DSS) for 7 days. The rhMG53 treatment group was intramuscularly injected with 1 mg MG53/kg body weight per day, and then replaced with single distilled water, ad libitum for 7 days. The body weight, disease activity index, and survival rate were observed for each of the groups.

The pathological score of colitis in mice with ulcerative colitis was taken from the colon tissue of mice (day 7 after DSS treatment). The tissue was embedded in paraffin, sectioned, and stained with HE. The degree of pathological damage was scored. The score was marked as: ulcer (Invalid 0 points, less than or equal to 3 mm for 1 point, greater than 3 mm for 2 points), inflammation (normal is 0 points, mild for 1 point, severe for 2 points), granulation tissue (not 0 points, there is 1 point) The depth of the lesion (normally 0 points, 1 point in the submucosa, 2 points in the muscular layer, 3 points in the serosa layer) and fibrosis (normal 0 points, mild 1 point, and severe 2 points).

The results of recombinant rhMG53 protein protection of DSS-induced ulcerative colitis model mice are depicted in FIGS. 3A-3C. The C57BL/6 mice were treated with 4% DSS to construct an acute IBD model. The animal model was evaluated by the colitis disease activity index, colitis pathological scoring standard and survival rate. Compared with the negative control group, the acute IBD model mice lost weight and the disease activity index increased (FIGS. 3A and 3B). Administration of exogenous rhMG53 (1 mg protein/kg body weight) revealed that, compared with the model control group, the weight of the mice treated with rhMG53 did not significantly decrease, the stool was formed, and no obvious bloody stool was observed. The data indicate that rhMG53 can effectively reduce the body weight disease activity index and increase the survival rate of DSS-induced acute IBD model mice. The data further indicate that MG53 provides good protective effect against ulcerative colitis.

Efficacy against ulcerative colitis was further established using colon length mouse model. The mouse colon tissue was measured for length from the ileocecal portion to the anus, and the results are shown in FIG. 4A and 4B. The results indicate that the colon shortened, the normal structure of the colonic pathological mucosa was destroyed, and the crypt structure was disordered (FIG. 4C). The pathological score of the colitis in the model group was significantly higher than that of the control group (FIG. 4D). The survival rate of the % DSS-induced ulcerative colitis model group was lower than that of the control group. This indicates successful induction of a mouse model of acute colitis. Compared with the untreated control group, the DAI of the colitis model mice was significantly increased on the third day, showing a decrease in body weight loss, watery stool, and acute moderate to severe colitis in the naked eye. The above results indicate that the rhMG53-treated group significantly inhibited colon length shortening in model mice. It can also be seen from FIG. 4C that the colonic tissue lesions of the DSS model group mainly involve the mucosal layer and the submucosa, and the inflammatory cell types are mainly mononuclear macrophages and neutrophils. In the severe area of inflammation, the local mucosal layer is completely necrotic and forms an ulcer. rhMG53 (1 mg protein/kg body weight) significantly reduced mononuclear macrophages and neutrophil infiltration in colonic tissue of model mice, and reduced mucosal necrosis and crypt disappearance.

### Example 4

### Evaluation of Efficacy in a Mouse Model for Crohn's Disease

BALB/c mice, male, 6-8 weeks old, weighing 22-25 g, were randomly divided into three groups: normal group, model group, and rhMG53 (1 mg/kg) treated group. Except for the normal control group, the other groups were intraperitoneally injected with 0.5% sodium pentobarbital anesthesia, and the flexible small tube was carefully inserted into the colon (3.5 cm proximal anus), and 2% 2,4,6-trinitrobenzenesulfonic acid (TNBS) was injected. 100 μL of benzenesulfonic acid was administered and the mice were inverted vertically for 1 minute. From the first day, rhMG53 (1 mg/kg) was administered intramuscularly, and the normal group and the 2,4,6-trinitrobenzenesulfonic acid model group were injected with an equal amount of 0.5% CMC-Na for 7 days. The body weight of the mice was weighed daily and the diarrhea and blood in the stool were observed. The results are depicted in FIGS. 5A and 5B.

As shown in FIG. 5B, the mice in the 2,4,6-trinitrobenzenesulfonic acid treated model group showed a significant decrease in body weight from day 2, diarrhea, fecal occult blood and blood in the stool, and gradually increased as the test continued. rhMG53 (1 mg/kg) effectively alleviated weight loss in mice, indicating that rhMG53 has a good protective effect on Crohn's disease model mice.

The survival rate of mice used in the Crohn's disease model was evaluated. FIG. 5A demonstrates that after 7 days of monitoring, the survival rate of the 2,4,6-trinitrobenzenesulfonic acid treated model group mice was only 50%, and rhMG53 (1 mg/kg) significantly increased the survival rate of the mice.

### Example 5

### Preparation of Bioengineered Lactobacillus Bacteria that Express and Secrete MG53

Experimental strains and plasmids: Food grade Lactococcus (lactic acid bacteria) NZ9000 and secretory expression vector pNZ8148 were purchased from the BioVector Plasmid Vector Culture Cell Genetic Collection Center. The full sequence of the human TRIM72 gene (MG53 protein-coding gene), signal peptide and Lactobacillus acidophilus is found in the NCBI gene pool, and the primer and human TRIM72 gene were provided by Shanghai Biotech Co., Ltd., and the SPUSp45 signal peptide sequence was increased upstream of the TRIM72 gene. The His gene sequence is specifically shown in SEQ ID NO. 1 and SEQ ID NO. 4. The plasmid small kit is Tiangen's product; Tricine and Nisin are Sigma products; Coomassie Brilliant Blue Kit is Biyuntian's product; MG53 antibody was kindly provided by Prof. Ma Jianjie (TRIM-edicine, Inc., Columbus, Ohio, USA).

The construction of food-grade expression vector pNZ8148 was ligated to the MG53 gene: the ligation site is depicted in FIG. 6A. The secretion expression vector pNZ8148 is ligated to the MG53 gene: the ligation site is shown as A in FIG. 6.
1) Reorganization connection
Treatment of pNZ8148 plasmid linearization vector with NcoI-KpnI:
Recombination reaction system:
The total volume of 10 ul was gently mixed in a 50° C. water bath for 25 min.
2) Conversion
10 μl of the ligation product was added to 50 μl of competent cells (Tiangen), ice bath for 30 min, 42° C. for 90 sec, and immediately ice bath for 2 min. The mixture was incubated with 150 μl of sterile LB medium and shaken at 37° C. for 1 hour. The cells were cultured in LB plates containing chloramphenicol.
3) Colony screening experiment
a. Pick a single colony in the overnight plate
b. Colony PCR using TRIM72 primers
c. Electrophoretic identification of positive clones
d. Four positive bacteria were randomly selected and cultured overnight with a 4 ml single tube shaker at 37° C.

TRIM72-seqF:
(SEQ ID NO. 2)
5′-caccgttctctgcccctg-3′
TRIM72-seqR:
(SEQ ID NO. 3)
5′-ctgtgtcttgaggcgtgc-3′

4) Extraction plasmid
The plasmid was extracted from the overnight bacterial solution in 3.2.5 (Tiangen Co., Ltd. plasmid extraction kit), and sent for sequencing. The result is shown in SEQ ID NO. 4. The correct plasmid was obtained and designated Pnz8148-SPusp45-TRIM72, and stored at −80° C. for later use.
Preparation and electroporation of lactic acid bacteria NZ9000:
1) 4M1 G/L-SGM17B medium was inoculated with 4 μl of lactic acid NZ9000 at a concentration of 1%, and cultured at 30° C. for 12 h;
2) Adding all the bacterial liquid obtained in the first step to 80 ml of fresh G/L-SGM17B medium, and culturing until OD600=0.3-0.35;
3) 4Centrifuge at 5000 g for 20 min at ° C., discard the supernatant;
4) The pellet was resuspended in 80 ml of 0.5 M sucrose/10% glycerol, centrifuged at 6000 g for 20 min at 4° C., and the supernatant was discarded;
5) The pellet was resuspended in 40 ml of 0.5 M sucrose/10% glycerol/0.5 mM EDTA, pre-cooled for 15 min, centrifuged at 6000 g for 20 min at 4° C., and the supernatant was discarded:
6) The pellet was resuspended in 20 ml of 0.5 M sucrose/10% glycerol, centrifuged at 6000 g for 20 min at 4° C., and the supernatant was discarded;
7) The pellet was resuspended in 80 ml of 0.5 M sucrose/10% glycerol, dispensed into 40 μl/tube, and stored at −80° C.
Recombinant sub-electricity:
1) The electric rotor was pre-cooled on ice and UV-sterilized in a clean bench.
2) The tube (40 μl/tube) was taken from a −80° C. refrigerator and thawed on ice.
The plasmid was removed from the −20° C. freezer and thawed on ice.
3) The Pnz8148-linked MG53 product was added to 40 ul of the competent state, and after flicking, the mixture was iced for 10 min.
4) Transfer the mixture from step 3 to a pre-cooled electric rotor to ensure that the mixture is at the bottom of the electric rotor and that no air bubbles are present.
5) Remove the condensate from the outer wall of the electric rotor and place the electric rotor into the electric shock meter.
6) Set the voltage of the electric shock meter to 2000V and the electric shock time to 4 ms.
7) Electric shock once.
Recombinant resuscitation and PCR identification:
1) After the shock is completed, quickly remove the electric rotor from the shock slot.
2) Add lg17MC regeneration medium to 1 ml ice bath.
3) Gently mix with a pipette, ice bath for 5 min, then transfer to 1.5 ml EP tube, and let stand for 1-1.5 h at 30° C.
4) 10 μl, 100 μl, and 900 μl were plated on a BCP plate at 30° C. for anaerobic standing for 1-2 days.
5) A single colony that turned yellow from the BCP plate was placed in 3 ml of GM17 liquid medium, and cultured overnight at 30° C.;
6) Preparation of bacterial solution template DNA: 200 μl of the bacterial solution was centrifuged in a 1.5 ml sterile EP centrifuge tube, centrifuged at 12,000 rpm, the medium was discarded, and the cells were resuspended by adding 100 μl of sterile ultrapure water. Then, the above EP tube was placed at −80° C. for 15 min, heated in a boiling water bath for 15 min, the cells were fully lysed, centrifuged at 12000 rpm for 3 min, and the supernatant was template DNA;
7) Take 1 μl of bacterial DNA as a template, MG53 as the upstream and downstream primers 0.3 μl each, the primer sequence as the upstream primer: 5′-caccgttctctgcccctg-3′ (SEQ ID NO. 2); downstream primer: 5′-ctgtgtcttgaggcgtgc-3′ (SEQ ID NO. 3), molecular weight: 250 bp. 102 μl of PCR buffer, 2 μl of dNTP solution and 0.2 μl of TaqDNA polymerase were denatured for 5 min and then entered into circulation. The cycle temperature and time were 94° C., 30 s of water was added to the total volume of 20 μl; after 94° C., 55° C., 74° C., 25 cycles. Then, it was further extended at 72° C. for 10 min; the product was detected by 1.0% agarose gel electrophoresis, and the results are shown in B of FIG. 6.
Target protein expression:
1) The recombinants identified as positive by PCR in 7 were inoculated into 3 ml of GM17 liquid medium according to the inoculation amount of 5%, and cultured at 30° C. for 48 h.
2) Take 2 ml of bacterial solution at 12000 rpm, 4° C., and centrifuge for 5 min;
3) Take 2 ml of the supernatant, add 200 μl of 150% (w/v) trichloroacetic acid (TCA), stand at 4° C. for 12 h in the dark, centrifuge at 12000 rpm, 4° C. for 10 min, and precipitate with 1 ml of pre-cooled acetone. Hanging; 12000 rpm, 4° C., centrifugation for 10 min, the pellet was resuspended in 1 ml of pre-cooled acetone; centrifuged at 12000 rpm, 4° C. for 10 min, carefully pipet the supernatant with a 1 ml pipette, retain the precipitate, and air dry, add 100 μl ddH2O and 20 μl loading buffer (6×), boiled for 10 min, 12000 rpm, room temperature, centrifuged for 10 min and then used;
4) The cells were resuspended in 500 μl of ddH 2 O, centrifuged at 12,000 rpm, room temperature for 5 min, and the supernatant was discarded. The cells were resuspended in 200 μl of pH (8.0) phosphate buffer (PBS), and 5 μl of lysozyme (50 mg/ml) was added, 37°. C water bath 1.5 h, add 20 μl SDS solution (20%), boil for 10 min in boiling water, add 50 μl loading buffer (6×), boil water for 10 min, 12000 rpm, room temperature, centrifuge for 10 min and then set aside.
5) An equal amount of total protein was electrophoresed in a 10% SDS polyacrylamide gel and transferred to a nitrocellulose membrane. The non-specific binding site was blocked with 5% skim milk powder at room temperature, and then the membrane was incubated with MG53 antibody (antibody dilution 1:800) at 4° C. overnight. The membrane was washed 3 times with TBST, and the goat anti-rabbit secondary antibody (diluted 1:10000) was added and incubated for 1 h at room temperature. After TBST was washed for 3 times, it was scanned by the Odys-sey two-color infrared fluorescence imaging system of LI-COR, USA. As a result, MG53 expression was found in the NZ9000-TRIM72 bacterial solution, whereas MG53 was not expressed in the NZ9000-VC (FIG. 6, C).

### Example 6

### MG53 Secreted by Lactobacillus Protects Against DSS-Induced Ulcerative Colitis and TNBS-Induced Crohn's Disease

The C57 mice maintained in a SPF animal facility at the Experimental Animal Center of Daping Hospital of the Army Military Medical University weighed 22 to 25 g. The surgical operation during the experiment was conducted in accordance with the regulations concerning experimental animals of the Army Military Medical University. Mice were randomly divided into four groups: normal group, model group (4% DSS), lactobacillus treatment group (engineered lactobacillus that secretes exogenous MG53), and Lactococcus lactis control group. Except for the normal control group and the Lactococcus lactis control group, the other two groups were free to drink 4% dextran sulfate sodium (DSS) for 7 days, and the exogenous MG53 Lactococcus lactis treatment group was given DSS daily and then switched to single distilled water and allowed to drink freely for 7 days. The effect MG53 upon the survival rate of mice was monitored, and the results are depicted in FIG. 7. The results indicated that the exogenous MG53 released by Lactococcus lactis could improve the survival rate of Crohn's disease model mice.

### Example 7

### Effect of Engineered Lactobacillus Lactis (that Secretes MG53) Upon TNBS-Induced Crohn's Disease

The C57 mice maintained in a SPF animal facility at the Experimental Animal Center of Daping Hospital of the Army Military Medical University weighed 22 to 25 g. The surgical operation during the experiment was conducted in accordance with the regulations of the experimental animals of the Army Military Medical University. Mice were randomly divided into four groups: normal group, model group (TNBS), lactobacillus treatment group (engineered lactobacillus that secretes exogenous MG53), and Lactococcus lactis control group. As depicted in FIG. 8, the mice in the 2,4,6-trinitrobenzenesulfonic acid model group showed a significant decrease in body weight from day 2, diarrhea, fecal occult blood and blood in the stool, and gradually increased as the test continued. The Lactococcus lactis treatment group of exogenous MG53 can effectively alleviate the weight loss of mice and improve the survival rate of Crohn's disease model mice.

### Example 8

### Oral Dosage Form of rhMG53 and EUDRAGIT S-100

rhMG53 is provided by TRIM-edicine, Inc. (Columbus, Ohio). EUDRAGIT S-100 (Poly(methacylic acid-co-methyl methacrylate) 1:2) is provided by EVONIK (https://healthcare.evonik.com/product/health-care/en/). The following procedure is used to prepare beads.

In a 100 mL beaker, add 35 mL water, and stir. While stirring add Eudragit S-100 powder (1.4 g), then and 12N NH₄OH (0.82 mL).

Add 2-hydroxypropyl)-β-cyclodextrin (0.24 g) to a 10 mL water (CD: 24 mg/mL).

Prepare a solution of MG53 (70 mg in ˜15.5 mL PBS) at a pH 8. To this solution, add 10 mL of the CD solution and 10 mL of water for a total volume of 35.55 mL.

Mix the MG53/CD solution with the Eudragit solution while stirring.

Spray dry the resulting suspension to form the powdered dosage form containing MG53 (70 mg), EUDRAGIT (1.4 g), salts (130 mg), and CD (0.24 g) for a total solids content of 1.77 g or a MG53-loading of 40 mg/g of solid (4% loading). Spray drying conditions used: nozzle size—0.6 mm; air speed—0.3 m³/min; air outlet temp: 38 C; room temperature: 24 C; room humidity: 53%.

The powder can be included in a capsule, caplet, tablet or other oral dosage form.

### Example 9

### Preparation of MG53-Containing Enteric Release Composition

The enteric release formulation comprises MG53, hydroxypropyl-beta-cyclodextrin (HP-b-CD), and methacrylic acid/methyl methacrylate anionic copolymer (EUDRAGIT S 100; dissolution in water at pH above 7.0). The following procedure was used.
1. In a 100 mL beaker, added 35 mL water;
2. weighed 1.4 g Eudragit S-100 powder and added to the H₂O above while stirring;
3. added 0.82 mL of 12N NH₄OH and stirred continuously for half an hour while periodically checking pH;
4. weighed 0.24 g of 2-hydroxypropyl)-O-cyclodextrin and added to a vial containing 10 mL water (CD: 24 mg/mL);
5. added 70 mg MG53 to 15.5 mL PBS at pH 8;
6. mixed the HP-b-CD solution with the MG53-containing solution and add water to a total volume of 35.55 mL;
7. added the MG53/CD solution to the 35 mL Eudragit-containing solution while stirring, and added 1 mL of water;
6. spray dried the MG53/CD/EUDRAGIT solution according to Example 11;

The resulting enteric release composition contained: MG53 70 mg; Eudragit 1.4 g; salts: 15.5/2*17 mg/mL=130 mg; CD 0.24 g.

### Example 10

### Determination of In-Vitro Release Profile

A known amount of powdered samples of MG53 containing enteric release composition (made according to Example 9) was dispersed in pH 2 (0.01N HCl) solution for 2 hrs, followed by addition of Na₃PO₄ solution (9.7 g in 112.8 mL H₂O; to pH 6.5), then followed by addition of same Na₃PO₄ solution to pH 7.5. At different time points, the released MG53 solution was centrifuged at 17,000 g for 20 min, filtered through 0.22 um filter, and the absorbance at 280 nm was measured (n=2 per time points). The release experiments were performed in an orbital shaker at 37° C. and 150 rpm.

### Example 11

### Spraying Drying of MG53/CD/EUDRAGIT Mixture

The MG53/CD/EUDRAGIT mixture was produced by spray-drying using the laboratory scale ProCept 4M8-TriX spray-dryer (Zelzate, Belgium). Drug-polymer solutions were prepared in the binary solvent mixture of interest DCM/EtOH 2:1 (v/v) at 50 mg/mL. The feed solution flow rate was adjusted at 5 g/min. An atomizing air pressure of 0.65 bars was applied to a 1.2 mm bifluid nozzle to create a spray. The drying gas airflow was set at 0.35 m3/min and maintained at 65° C. The lateral cooling air was kept constant at 100 L/min and dried particles were separated from the exhaust air within the medium cyclone (height/diameter of 242 mm/60 mm). After processing, the spray-dried material was stored in a vacuum oven for 48 h before analysis to eliminate the last traces of residual solvent.

### Example 12

### Evaluation of MG53-Containing Enteric Formulation

The formulation of Example 9 was administered daily to mice by oral gavage: dose of rhMG53 equivalent to 200 microg/dose. The mice were subjected to DSS-induced IBD as described herein. The body weight, GI integrity (FITC-dextran level), and colon length were determined as described herein.

### Example 13

### Evaluation of Fluorescence-Labeled Enteric Formulations

A fluorescence labeled-MG53 (FITC-MG53) was prepared according to well-known procedure and then encapsulated with EUDRAGIT according to Example 9, wherein FITC-MG53 was used in place of native MG53. The fluorescence label (FITC) was also encapsulated with EUDRAGIT according to Example 9, wherein FITC was used in place of native MG53.

Mice were administered the encapsulated FITC and FITC-MG53 by oral gavage at seven days post DSS treatment. Colon tissue was resected from control (untreated) mice, FITC-treated mice and FITC-MG53-treated mice. Confocal microscopy imaging revealed targeting of FITC-MG53 to the injured intestinal epithelia layer. FITC vehicle alone did not show targeting at the epithelia layer (FIG. 13).

### Example 14

### Evaluation of MG53 for Protection of Enteroids

Enteroids were formed from primary intestinal stem cells derived from C57BL6/J mice and cultured for 1 week. Treatment of enteroids with DSS for 48 hours led to the reduction of budding enteroids. rhMG53 (10 μg/mL) significantly increased the number of budding enteriods indicating the ability of MG53 to protect stem cells from DSS-induced damage.

### Example 15

### Preparation of Food-Grade Engineered MG53-Expressing Lactobacillus

The genetic code of MG53 protein was optimized for expression in Lactobacillus paracasei. The maltose-induced promotor Pₘₐₗ, signal peptide SP_{H5} from the surface protein slpH5, and mg53 gene were composed of MG53 gene expression element (PS53). The upstream fragment (UF, 673 bp) and the downstream fragment (DF, 764 bp) flanking upp gene (encoding uracil phosphoribosyltransferase, used as counterselectable genetic marker) in the chromosomal DNA were amplified and purified by gel recovery kit, respectively. The two fragments were successively fused with PS53 by overlap PCR to produce UF-PS53-DF fragment, which was ligated into temperature-sensitive plasmid pMG36 cm (containing chloramphenicol resistance gene cat) by Gibson assembly. The resulting recombinant plasmid, pMG3653, was electroporated into L. paracasei 27092. The correct transformants were propagated for 30 generations in MRS medium with chloramphenicol (10 μg/mL) at 30° C. and the single-crossover clones were selected by colony PCR. The single-crossover strains were subsequently propagated in MRS medium (without chloramphenicol) with 5-fluorouracil (20 μg/mL) and the concentration of 5-fluorouracil gradually increased to 150 μg/mL to induce double-crossover integration at 42° C. After 20 generations, the cultures were diluted and spread on MRS plates. The signal clones were simultaneously streaked on MRS plates with 5-fluorouracil (150 μg/mL) and with chloramphenicol (10 μg/mL) and the clones with 5-fluorouracil-resistance and chloramphenicol-sensitive phenotypes were the potential double-crossover strains. The chromosomal DNA fragments (about 4.0 kb) of the recombinant regions in the potential strains were amplified and sequenced to confirm the integration of PS53 fragment. The chromosomal DNA maps of the wild type (WT) and the recombinant strain (M1) are depicted in FIG. 16.

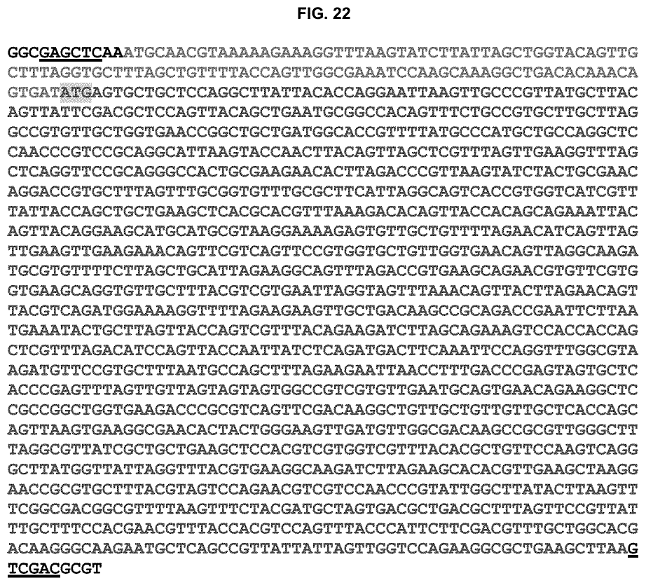

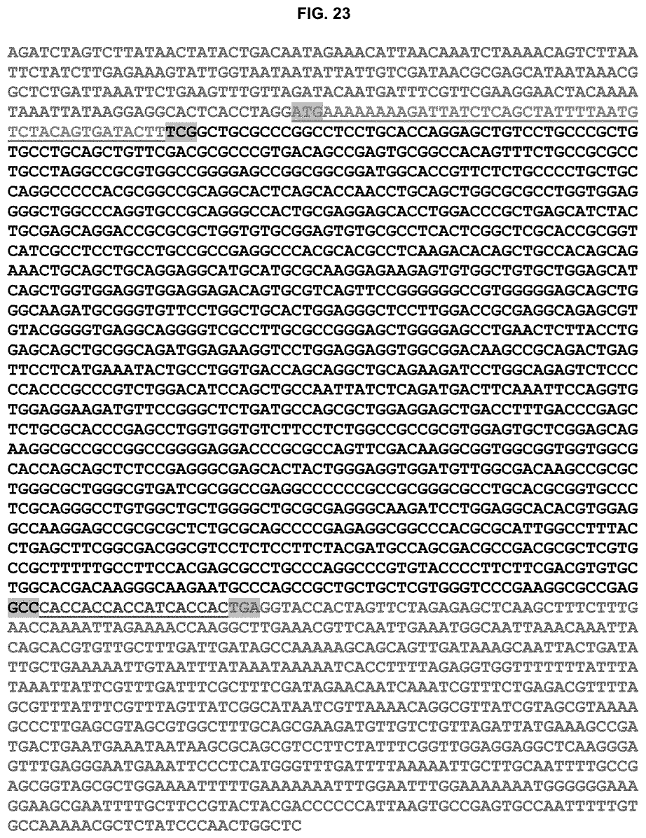

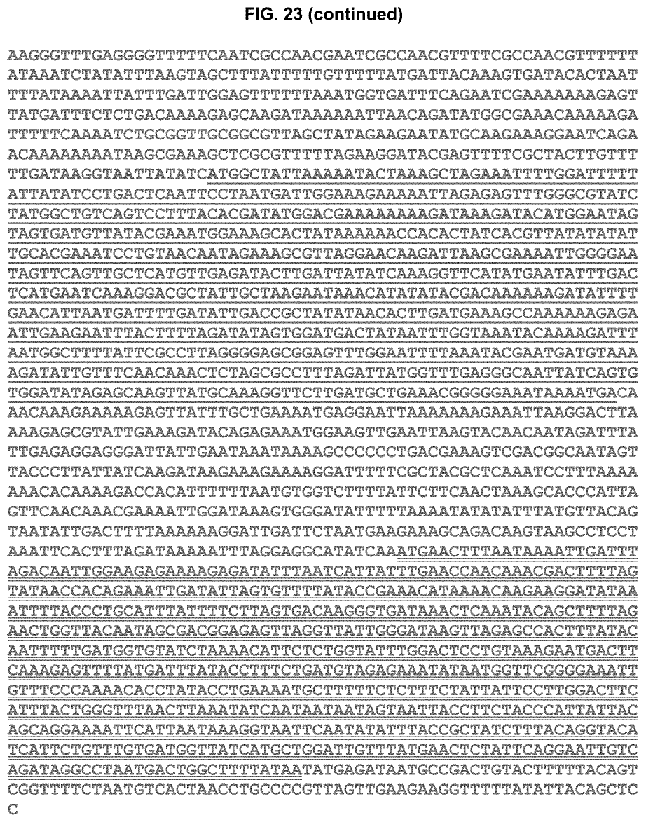

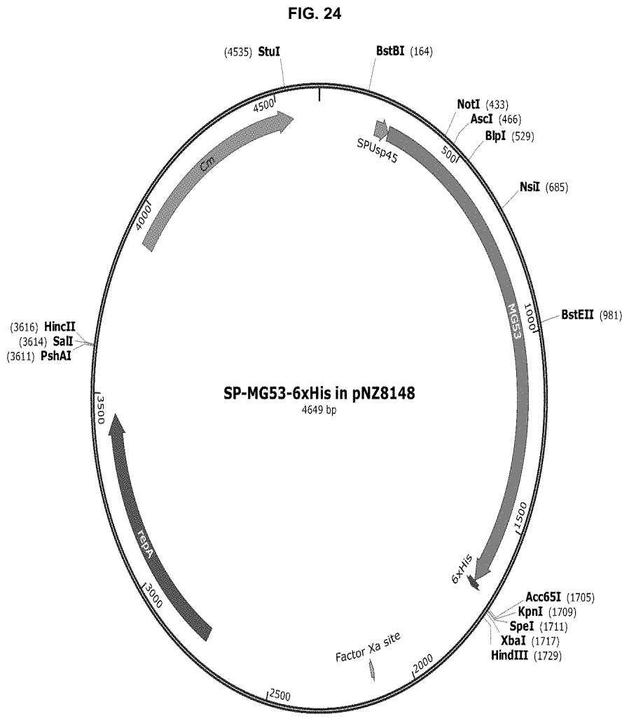

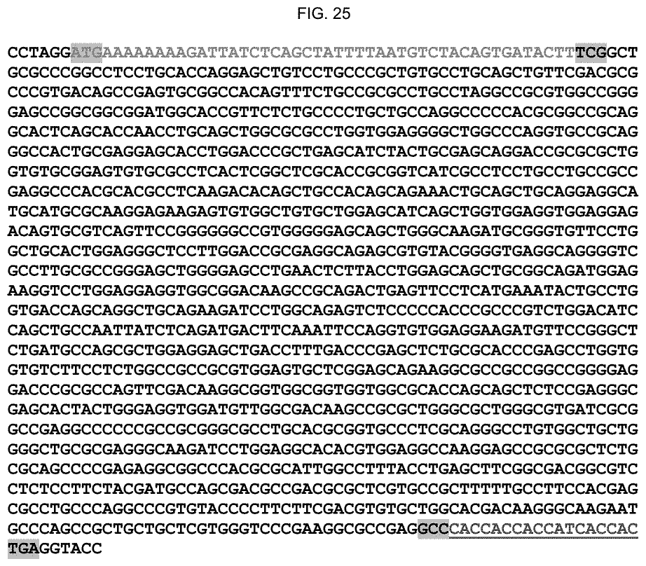

The M1 strain was cultured in MRS medium at 37° C. until stationary phase (about 24 hours) and then the cells were harvested and washed by MRS medium without glucose (MRSm medium). The above cells were propagated in MRSm medium with 1% maltose and 0.1 mol/L phosphate buffer (pH 7.4) for about 18 hours and the supernatant of the culture was collected by centrifugation. The proteins in the supernatant were precipitated by organic solution and detected by western blot. Secretion of MG53 from the engineered Lactobacillus was confirmed by Western blot (FIG. 17). A first gel depicts protein concentration versus signal was used to estimate the relative productivity of the engineered bacteria. The DNA sequence (SEQ ID NO. 5) for the expression module of MG53 is depicted in FIG. 22.

The signal peptide SP-prtP (PrtP cell wall associated protease) has the following sequences: protein (SEQ ID NO. 6) MQRKKKGLSILLAGTVALGALAVLPVG EIQAKADTNSD; DNA (SEQ ID NO. 7) ATGCAACGTAAAAAGAAAGGTT TAAGTATCTTATTAGCTGGTACAGTTGCTTTAGGTGCTTTAGCTGTTTTACCAGT TGGCGAAATCCAAGCAAAGGCTGACACAAACAGTGAT.

The signal peptide SP-slpA (SplA, cell surface protein) has the following sequences: protein (SEQ ID NO. 8) MKKNLRIVSAAAAALLAVAPVAASAVSVNAAATTTN; DNA (SEQ ID NO. 9) ATGAAGAAAAATTTAAGAATTGTTAGCGCTGCTGCTGCTGC TTTATTAGCTGTTGCTCCAGTTGCTGCTTCAGCTGTTTCTGTTAACGCTGCTGCA ACCACTACCAAC.

### Example 16

### Evaluation of Engineered MG53-Expressing Lactobacillus for the Treatment of IBD

Efficacy of engineered MG53-expressing Lactobacillus (Example 15) for the treatment of IBD was evaluated in mice with DSS-induced IBD. The engineered Lactobacillus (Lacto MG53) was compared to the native Lactobacillus (Lacto Co) and control (no treatment). Mice were administered the lactobacillus by oral gavage at seven days post DSS treatment. Bodyweight and FITC-dextran concentration in serum were determined as described herein.
1. 10 Male C57BL/6J mice (12 wk old) were randomly assigned to two groups and added 3.0% (w/v) DSS in the drinking water ad libitum for the first 5 days, then switch to 1% DSS till day 26.
2. Each mouse was given 250 mg (in 200 ul PBS) Control (native) Lactobacillus or MG35-expressing Lactobacillus by oral gavage every other day since day 6. (MG53˜125 ng/mouse)
3. At the end of the schedule, the mice were water starved for 4 hr. FITC-dextran was dissolved in PBS at a concentration of 100 mg/ml and administered to each mouse (40 mg/100 g body weight) by oral gavage.
4. After 4 hr, blood was collected by cheek punch from each mouse. The blood was held for 30 min at room temperature (RT), then centrifuged at 8,000 rpm for 10 min and serum was collected. After that, the serum was diluted with an equal volume of PBS and 100 μl aliquots of diluted serum were placed in a 96-well microplate in duplicate.
5. The concentration of FITC in serum was determined by spectrophotofluorometry with an excitation of 485 nm (20 nm band width) and an emission wavelength of 528 nm (20 nm band width) using as standard serially diluted FITC-dextran (0, 250, 500, 1,000, 2,000, 4,000, 8,000, 10,000 ng/ml) as standard. Serum from mice not administered with FITC-dextran was used to determine background.
6. After 6 days, mice were euthanized and vital organs collected to compare intestine length and collect Peyer's patches.

The data (FIGS. 18 and 19) demonstrate efficacy of the engineered lactobacillus. As compared to control, the native Lactobacillus provided insignificant reduction in weight loss (FIG. 18) and insignificant changes in FITC-dextran level in serum (FIG. 19). As compared to control, the engineered Lactobacillus provided significant reduction in weight loss (FIG. 18) and significant decrease in FITC-dextran level in serum (FIG. 19).

### Example 17

### Evaluation of MG53 for the Treatment of X-Ray Irradiation-Induced Intestinal Injury

C57BL6/J mice (both male and female) were irradiated by X-ray (10-30 Gy/day, IR) for a total of 6 days. Control mice injected with saline without IR. IR mice group receive X-ray and injected with saline. rhMG53 mice group receive X-Ray and tail vein injection of rhMG53 (1 mg/kg) at 15 min before IR.

At 6 days post IR, mice were sacrificed and the colon length measured. IR exposure caused shortening of the colon, which was partially mitigated by rhMG53 treatment (FIG. 20).

FITC-dextran measurement show disrupted GI integrity in mice receiving IR exposure (as indicated by the >500 fold elevation of FITC-dextran measured in the serum; FIG. 21). Mice receiving rhMG53 displayed reduced FITC-dextran in the serum, demonstrating the improved integrity of the GI in mice following IR exposure.

### Example 18

### Western Blot

Protein lysates from indicated tissue and cell sources were separated by SDS-PAGE. Proteins were transferred from gels to PVDF membranes at 4° C. The blots were washed with PBST (PBS+0.5% Tween-20), blocked with 5% milk in PBST for 2 hours, and incubated with indicated primary antibodies overnight at 4° C. under rotation. Secondary antibodies, anti-mouse or anti-rabbit IgG HRP conjugated, were applied at 1:5000 dilution and incubated for approximately 1.5 hours with shaking at room temperature. Immunoblots were visualized with an ECL plus kit (Pierce). The antibodies used in this study were as follows: rabbit anti-MG53 antibody was generated by our laboratory and the sensitivity and specificity were previously confirmed: anti-p-Smad2 antibody (Cell Signaling Technology, Cat. No. 3108); anti-Smad2 antibody (Cell Signaling Technology, Cat. No. 5339); anti-Smad5, (Cell Signaling Technology, Cat. No. 12534); anti-p-Smad5 antibody (Cell Signaling Technology, Cat. No. 9516); anti-GAPDH antibody (Cell Signaling Technology, Cat. No. 2118s); anti-alpha-SMA antibody (Invitrogen, Cat. No. 14-9760-82); and anti-fibronectin antibody (Sigma-Aldrich, Cat. No. F3648).

All data are expressed as mean±S.D. Groups were compared by Student's t test and analysis of variance for repeated measures. A value of p<0.05 was considered significant.

For any range herein, the upper and lower limits thereof are considered as being part of the range. Moreover, all integer and fractional values within said ranges are also considered as being within said range. Accordingly, all integers and fractional values within each specified range are hereby incorporated by reference.

All values disclosed herein may have standard technical measure error (standard deviation) of ±10%. The term “about” or “approximately” is intended to mean±10%, ±5%, ±2.5% or ±1% relative to a specified value, i.e. “about” 20% means 20±2%, 20±1%, 20±0.5% or 20±0.25%. The term “majority” or “major portion” is intended to mean more than half, when used in the context of two portions, or more than one-third, when used in the context of three portions. The term “minority” or “minor portion” is intended to mean less than half, when used in the context of two portions, or less than one-third, when used in the context of three portions. It should be noted that, unless otherwise specified, values herein concerning pharmacokinetic or dissolution parameters are typically representative of the mean or median values obtained.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

1. A method of treating and/or preventing IBD or radiation induced intestinal injury comprising administering to a subject an effective amount of a) a composition comprising MG53; b) a composition comprising live MG53-expressing microbe; or c) a probiotic composition comprising live MG53-expressing microbe.

2. The method of claim 1, wherein said composition is an enteric release composition, probiotic composition, food supplement, health supplement, healthcare product, dosage form, medicament, food, or food additive.

3. The method of claim 1, wherein said method is a method of reducing the occurrence of and/or reducing the severity of symptoms associated with IBD or radiation induced intestinal injury.

4. The method of claim 3, wherein said composition is an enteric release composition, probiotic composition, food supplement, health supplement, healthcare product, dosage form, medicament, food, or food additive.

5. A probiotic composition comprising a safe and non-antigenic MG53-expressing probiotic microbe that expresses MG53 in the GI tract of a subject after said subject is orally administered said probiotic microbe.

6. The probiotic composition of claim 5, wherein said MG53-expressing probiotic microbe is genetically-modified probiotic microbe selected from the group consisting of genetically-modified bacteria, genetically-modified yeast, or combination thereof.

7. The probiotic composition of claim 6, wherein said genetically-modified probiotic microbe comprises an MG53-gene expression vector, and said microbe expresses and secretes human MG53 protein.

8. The probiotic composition of claim 5, wherein said composition is an enteric release composition, food supplement, health supplement, healthcare product, dosage form, medicament, food, or food additive.

9. A method of forming MG53 in the gastrointestinal tract of a subject, the method comprising administering to said subject the probiotic composition of claim 5.

10. The method of claim 9 further comprising administering to said subject exogenous MG53.

11. The method of claim 9, wherein said composition is an enteric release composition, food supplement, health supplement, healthcare product, dosage form, medicament, food, or food additive.
